SEQUENCE LISTING (20 sequences)

SEQ ID NO: 1 - DNA, 73 bp, Artificial sequence
  Synthetic oligonucleotide
atcgcccgggaaaaattgaaattttattttttttttttggaatataaataaccatgcagatcccacaggcgcc

SEQ ID NO: 2 - DNA, 33 bp, Artificial sequence
  Synthetic oligonucleotide
atcggaattctcaggtttggaactggccggctg

SEQ ID NO: 3 - DNA, 504 bp, Artificial sequence
  Synthetic oligonucleotide
atgcagatcccacaggcgccctggccagtcgtctgggcggtgctacaactgggctggcggccaggatggttcttagactccccagacaggccctggaacccccccaccttctccccagccctgctcgtggtgaccgaaggggacaacgccaccttcacctgcagcttctccaacacatcggagagcttcgtgctaaactggtaccgcatgagccccagcaaccagacggacaagctggccgctttccccgaggaccgcagccagcccggccaggactgccgcttccgtgtcacacaactgcccaacgggcgtgacttccacatgagcgtggtcagggcccggcgcaatgacagcggcacctacctctgtggggccatctccctggcccccaaggcgcagatcaaagagagcctgcgggcagagctcagggtgacagagagaagggcagaagtgcccacagcccaccccagcccctcacccaggccagccggccagttccaaacc

SEQ ID NO: 4 - protein, 168 aa, Artificial sequence
  Synthetic polypeptide
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQT

SEQ ID NO: 5 - protein, 168 aa, Artificial sequence
  Synthetic polypeptide
MQIPQAPWPVVWAVLQLGWRPGWFLDSPDRPWNPPTFSPALLVVTEGDNATFTCSFSNTSESFVLNWYRMSPSNQTDKLAAFPEGRSQPGQDCRFRVTQLPNGRDFHMSVVRARRNDSGTYLCGAISLAPKAQIKESLRAELRVTERRAEVPTAHPSPSPRPAGQFQT

SEQ ID NO: 6 - protein, 153 aa, Artificial sequence
  Synthetic polypeptide
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 7 - protein, 219 aa, Artificial sequence
  Synthetic polypeptide
MCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 8 - protein, 328 aa, Artificial sequence
  Synthetic polypeptide
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 9 - protein, 164 aa, Artificial sequence
  Synthetic polypeptide
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHFDPRDVVSNINVFVLELKGSETTFMCEYADETATIVEFLNRWITFCQSIISTLTAAAHHHHHHHH

SEQ ID NO: 10 - DNA, 3042 bp, Artificial sequence
  Synthetic oligonucleotide
  misc_feature (1)..(10): n is a, c, g, or t
  misc_feature (12)..(12): n is a, c, g, or t
  misc_feature (15)..(15): n is a, c, g, or t
  misc_feature (18)..(18): n is a, c, g, or t
  misc_feature (622)..(624): n is a, c, g, or t
  misc_feature (1391)..(1391): n is a, c, g, or t
  misc_feature (1860)..(1860): n is a, c, g, or t
nnnnnnnnnncncgnggnggcggccgctctagtaggattacctggtctatatagataacaaaacctacgtacgtataaacgagaccgttgtaccggagaacgagtatctggcagcgaaggccccgcgagtgacctgtttccacacggacttgatccccattacggacgaagagacacaacgacgttttgagaaaatgattgtacaggcggcgttagaggacgccctaacgagcatctttgaggagcacgacaataacgtaaccgattacttcgcggaatacatgcgatccctccaaatggcgaataaaagtcatacgaataatattatcgcggtcgctttagcggggataatcgtcattgtaacgacctacgtgtttactagattacgcactaagcaaaaaaaaggaaattataacgtacgtaataagatagataattccatacagaaagagattcagttggacggtgtatatactactgacaacgtttttatataaacatggtgtttatatttattatcacctgtgtatgtttggtgacgagatcctgtgggggtgggttagaagacgatatagatcgcatatttcaaaaacgatacaacgaactgagccagccgattaannncaatatgcgtacactgtgcaagtttagaggaattaccgcgactatgtttacggaaggagaatcttaccttattcaatgtcccataattcacgattacgtgctacgggcgctgtatgacttagtggaaggaagttacacggtacgctgggaacgcgaaacggaagacgatgttgagtcggtagatccgaagttagtcaaagggacgctattatacctccaacctaacgcgtccagtataggaacgtatctatgtaccttacacgataaccgaggtatgtgttatcaatctgtcgcgcacgtcatccgacgtccgaagatgcaatgcgtgaaacatgcacatacgacatcggacagcaacctgtggatatacctcgccattttagcagttttgatatccttaggcgtcctgtaaaggaaacgcgccagactccggaactatgaaggatttatcactgtatacagactccgacgtacgaaggataatcacgacgtaactcgaactctgcaggtcgactctagaggatctactagtcatatggatttaaaaatagcggagcttaaaaattgaaattttattttttttttttggaatataaataagctcgaagtcgacagatctaggcctggtacccgatccaccggtcgccaccatggtgagcaagggcgaggagctgttcaccggggtggtgcccatcctggtcgagctggacggcgacgtaaacggccacaagttcagcgtgtccggcgagggcgagggcgatgccacntacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgntgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgggtaattacccgggatgttttcaggtcttaccctcaactgtgtcctgctgctgctgcaactactacttgcaaggtcattggaaaatgcttatgtgtttgaggttggtaagaatgcctatctgccctgcagttacactctatctacacctggggcacttgtgcctatgtgctggggcaagggattctgtccttggtcacagtgtaccaacgagttgctcagaactgatgaaagaaatgtgacatatcagaaatccagcagataccagctaaagggcgatctcaacaaaggagacgtgtctctgatcataaagaatgtgactctggatgaccatgggacctactgctgcaggatacagttccctggtcttatgaatgataaaaaattagaactgaaattagacatcaaagcagccaaggtcactccagctcagactgcccatggggactctactacagcttctccaagaaccctaaccacggagagaaatggttcagagacacagacactggtgaccctccataataacaatggaacaaaaatttccacatgggctgatgaaattaaggactctggagaaacgatcagaactgctatccactaggaattctaacattttttaaaacaatttcgttatgttaaattatggaacggtcgcccacttacacggtacacgataaacgcttttctatcgtcgcactaaacggacaatacgacatggtggacgattttggtcttagtttttcttacacagcgatcgacgatatttctaaaaatcattccatcaaacacgttttagaagaatacttttcatggcgcgcgtatataggccgggtatgtatcataccgaatcacgtgggaaagctctacatcaaacttacaaagttggacaccacggcgaagaacaaactaggcaatctagatatattgttatgcgacgtgttaaaaatagacgaggacggaggcaacgagaaactgtttcaattcatacggtcgcggatcccccaattcgatatcaagcttatcgataccgtcgacctcg

SEQ ID NO: 11 - DNA, 585 bp, Artificial sequence
  Synthetic oligonucleotide
atgttttcaggtcttaccctcaactgtgtcctgctgctgctgcaactactacttgcaaggtcattggaaaatgcttatgtgtttgaggttggtaagaatgcctatctgccctgcagttacactctatctacacctggggcacttgtgcctatgtgctggggcaagggattctgtccttggtcacagtgtaccaacgagttgctcagaactgatgaaagaaatgtgacatatcagaaatccagcagataccagctaaagggcgatctcaacaaaggagacgtgtctctgatcataaagaatgtgactctggatgaccatgggacctactgctgcaggatacagttccctggtcttatgaatgataaaaaattagaactgaaattagacatcaaagcagccaaggtcactccagctcagactgcccatggggactctactacagcttctccaagaaccctaaccacggagagaaatggttcagagacacagacactggtgaccctccataataacaatggaacaaaaatttccacatgggctgatgaaattaaggactctggagaaacgatcagaactgctatccac

SEQ ID NO: 12 - DNA, 177 bp, Artificial sequence
  Synthetic oligonucleotide
cttgtgctctttggggcaggattcggcgcagtaataacagtcgtcgtcatcgttgtcatcatcaaatgcttctgtaagcacagaagctgtttcagaagaaatgaggcaagcagagaaacaaacaacagccttaccttcgggcctgaagaagcattagctgaacagaccgtcttcctt

SEQ ID NO: 13 - DNA, 7620 bp, Artificial sequence
  Synthetic oligonucleotide
agcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggagctccaccgcggtggcggccgcataaacgcgtttaaacagtcccccgtacgcggtacatcgtacgcacacttcactaacgatgtcgtacatcgattacacaaagaagtagagtcatacgacgtacgtttccctataaaatcggtaaacctagacgcggtgtttctatccataaacgtaacacgtgtacgtctacgttggaagatacccttgaccgaacacaatccttatcagacggcctacggatgttctaacgacagattatacagctacaacgagtacgctttttctcatttaaaacaagaccgtgtaaagatcatagaactcccatgtgacgacgattacagcgtcgtgttaatcacacacgatagccgttcgactattacaccggataaagtgaccgggtggctgcgcacgacccgtctacgttacgtaaacgtatccctacccaagggttccacggaaacgggacacaacgtaacgtgtctaactcccacacacgtcaatctatgtcatcgttgtcgtataacgattaccaaaacgggcgtggacgcaaccgcgttctcatgcgtcgacggcgatacatgcaccgaacacgacacgaccgcgtcaacgtgtacgattattataaaaacgacgggactggactttttgtttatggggaaactctaaaaaaaattgtcaattaaagtaactgcagatcgatcgcatatgaaaattgaaattttattttttttttttggaatataaataatggtgagcaagggcgaggaggtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctccaagacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctggggcatggcaccggcagcaccggcagcggcagctccggcaccgcctcctccgaggacaacaacatggccgtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctccaagacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctgtacggcatggacgagctgtacaagtaacccgggaaaaattgaaattttattttttttttttggaatataaataaccatgtgtcctcagaagctaaccatctcctggtttgccatcgttttgctggtgtctccactcatggccatgtgggagctggagaaagacgtttatgttgtagaggtggactggactcccgatgcccctggagaaacagtgaacctcacctgtgacacgcctgaagaagatgacatcacctggacctcagaccagagacatggagtcataggctctggaaagaccctgaccatcactgtcaaagagtttctagatgctggccagtacacctgccacaaaggaggcgagactctgagccactcacatctgctgctccacaagaaggaaaatggaatttggtccactgaaattttaaaaaatttcaaaaacaagactttcctgaagtgtgaagcaccaaattactccggacggttcacgtgctcatggctggtgcaaagaaacatggacttgaagttcaacatcaagagcagtagcagttcccctgactctcgggcagtgacatgtggaatggcgtctctgtctgcagagaaggtcacactggaccaaagggactatgagaagtattcagtgtcctgccaggaggatgtcacctgcccaactgccgaggagaccctgcccattgaactggcgttggaagcacggcagcagaataaatatgagaactacagcaccagcttcttcatcagggacatcatcaaaccagacccgcccaagaacttgcagatgaagcctttgaagaactcacaggtggaggtcagctgggagtaccctgactcctggagcactccccattcctacttctccctcaagttctttgttcgaatccagcgcaagaaagaaaagatgaaggagacagaggaggggtgtaaccagaaaggtgcgttcctcgtagagaagacatctaccgaagtccaatgcaaaggcgggaatgtctgcgtgcaagctcaggatcgctattacaattcctcatgcagcaagtgggcatgtgttccctgcagggtccgatccggtggcggtggctcgggcggtggtgggtcgggtggcggcggatctagggtcattccagtctctggacctgccaggtgtcttagccagtcccgaaacctgctgaagaccacagatgacatggtgaagacggccagagaaaaactgaaacattattcctgcactgctgaagacatcgatcatgaagacatcacacgggaccaaaccagcacattgaagacctgtttaccactggaactacacaagaacgagagttgcctggctactagagagacttcttccacaacaagagggagctgcctgcccccacagaagacgtctttgatgatgaccctgtgccttggtagcatctatgaggacttgaagatgtaccagacagagttccaggccatcaacgcagcacttcagaatcacaaccatcagcagatcattctagacaagggcatgctggtggccatcgatgagctgatgcagtctctgaatcataatggcgagactctgcgccagaaacctcctgtgggagaagcagacccttacagagtgaaaatgaagctctgcatcctgcttcacgccttcagcacccgcgtcgtgaccatcaacagggtgatgggctatctgagctccgcccttgtgctctttggggcaggattcggcgcagtaataacagtcgtcgtcatcgttgtcatcatcaaatgcttctgtaagcacagaagctgtttcagaagaaatgaggcaagcagagaaacaaacaacagccttaccttcgggcctgaagaagcattagctgaacagaccgtcttcctttgagaattcacgaatcgaataaaaacccgtgtacacacggacgttaattttttttgtggtttaaaaaatgaccacatttacgcttttttttaacgcgttatataaggtatctcgtttgtctataacaaagatcgtaactgaccttttttatatcgagaaaacatacgtttagttcatcctcaaacgtaacaccgtaactgcctcggacatcctccttgttgtcgtacacaaacatactaatcggatgcgtgaaatgaggattcactttaatcggattggtttctaggttaacacatgttacacaagatcctaagatggttatggacacatccttgttgtgatgtaacgagtcgggaagttgattgccgtagttgcccacgtcgccctccggttccagacacgtaatggttaggtatatatccgaatacttcgtcaacggatgagtcgtaaataacatgatggatagcttgttcccatctcctgcaccagcactggccgccacaaatcgttgtaccacgttagtaatcgtaatgtttatcataagcccgtacccggttaatatgagcgtggacgttttatgatcgtatcgttccttcatgtgacattctcccataaccgtttcgacgtaccgatttaacccgatggttagctcggcggctaagtgccagtggatcccccaattcgatatcaagcttatcgataccgtcgacctcgagggggggcccggtacccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaag

SEQ ID NO: 14 - protein, 26 aa, Homo sapiens
NQTDKLAAGPEDRSQPGQDCRFRVTQ

SEQ ID NO: 15 - protein, 26 aa, Mus musculus
NQTEKQAAFCNGLSQPVQDARFQIIQ

SEQ ID NO: 16 - protein, 26 aa, Rattus rattus
NQTEKQAAFCNGYSQPVRDARFQIVQ

SEQ ID NO: 17 - protein, 26 aa, Canis familiaris
NQTDKLAAFQEDRIEPGRDRRFRVMR

SEQ ID NO: 18 - protein, 26 aa, Equus caballus
NQTDKLAAFREDSSQPGRSGRFRVTR

SEQ ID NO: 19 - protein, 195 aa, Artificial sequence
  Synthetic polypeptide
MGSGLTLNCVLLLLQLLLARSLEDGYKVEVGKNAYLPCSYTLPTSGTLVPMCWGKGFCPQSQCTNELLRTDERNVTYQKSSRYQLKGDLNKGDVSLIIKNVTLDDHGTYCCRIQFPGLMNDKKLELKLDIKAAKVTPAQTAHGDSTTASPRTLTTERNGSETQTLVTLHNNNGTKISTWADEIKDSGETIRTAIH

SEQ ID NO: 20 - DNA, 39 bp, Artificial sequence
  Synthetic oligonucleotide
aaaattgaaattttattttttttttttggaatataaata

(19) United States
(12) Patent Application Publication
(10) Pub. No.: US 2021/0196771 A1
(43) Pub. Date: Jul. 1, 2021

(54) RECOMBINANT MYXOMA VIRUSES AND USES THEREOF

(71) Applicants: SYSTEMS ONCOLOGY, LLC, Scottsdale, AZ (US); MUSC FOUNDATION FOR RESEARCH DEVELOPMENT, Charleston, SC (US)

(72) Inventors: Jeff KIEFER, Scottsdale, AZ (US); Ramon MORENO, Scottsdale, AZ (US); Spyro MOUSSES, Scottsdale, AZ (US); Eric BARTEE, John's Island, SC (US)

(73) Assignees: SYSTEMS ONCOLOGY, LLC, Scottsdale, AZ (US); MUSC FOUNDATION FOR RESEARCH DEVELOPMENT, Charleston, SC (US)

(21) Appl. No.: 17/268,656

(22) PCT Filed: Aug. 16, 2019
(86) PCT No.: PCT/US2019/046823

§ 371 Date: Feb. 16, 2021

Related U.S. Application Data
(60) Provisional application No. 62/813,375, filed on Mar. 4, 2019.
(60) Provisional application No. 62/754,622, filed on Nov. 2, 2018.
(60) Provisional application No. 62/741,404, filed on Oct. 4, 2018.
(60) Provisional application No. 62/718,990, filed on Aug. 16, 2018.

(51) Int. Cl. A61K 35/768; C07K 14/705; C07K 14/54; C07K 14/55; A61K 45/06; C12N 7/00; A61P 35/00

(52) U.S. Cl. CPC A61K 35/768; C07K 14/70521; C07K 14/5434; A61P 35/00; A61K 45/06; C12N 7/00; C07K 14/55

(57) ABSTRACT

The present disclosure provides a recombinant oncolytic virus engineered to express a soluble form of an immune checkpoint protein. In certain aspects, the oncolytic vims is a replication competent virus such as myxoma vims. Methods of cancer treatment comprising administering the recombinant oncolytic virus expressing the soluble form of the immune checkpoint protein are also provided.

This application claims the benefit of U.S. Provisional Patent Application Nos. 62/718,990, filed Aug. 16, 2018; 62/741,404, filed Oct. 4, 2018; 62/754,622, filed Nov. 2, 2018; and 62/813,375, filed Mar. 4, 2019, the entirety of each of which are incorporated herein by reference.

The invention was made with government support under Grant No. 5R01CA194090 awarded by the National Institute of Health. The government has certain rights in the invention.

### BACKGROUND

### 1. Field

The present disclosure relates generally to the field of molecular biology and medicine. More particularly, it concerns oncolytic viruses expressing an immune checkpoint protein, such as PD1 or TIM3.

### 2. Description of Related Art

Current treatments used to treat various types of cancer tend to work by poisoning or killing the cancerous cell. Unfortunately, treatments that are toxic to cancer cells typically tend to be toxic to healthy cells as well. Moreover, the heterogeneous nature of tumors is one of the primary reasons that effective treatments for cancer remain elusive. Current mainstream therapies such as chemotherapy and radiotherapy tend to be used within a narrow therapeutic window of toxicity. These types of therapies have limited applicability due to the varying types of tumor cells and the limited window in which these treatments can be administered. Modern anticancer therapies currently being developed attempt to selectively target tumor cells while being less toxic to healthy cells, thereby being more likely to leave healthy cells unaffected.

Metastatic melanoma is an aggressive disease with a 16% 5-year survival rate and responds poorly to most standard chemotherapies. Interferon and interleukin 2 (IL-2) have both been approved by the U.S. Food and Drug Administration for the treatment of melanoma. Both mediate their benefit by stimulating an antitumor immune response. However, toxicity and low response rates have limited their use significantly. The first immune-checkpoint inhibitor approved by the U.S. Food and Drug Administration (FDA) was ipilimumab, a fully human immunoglobulin G1 monoclonal antibody that blocks cytotoxic T-lymphocyte antigen (CTLA)-4 and consequently the PD-1 pathway for the treatment of metastatic melanoma in 2011. The finding that programmed cell death protein 1 ligand 1 (PDL1 or PD-L1) and PDL2 are expressed by melanoma cells, T cells, B cells and natural killer cells led to the development of programmed cell death protein 1 (PD1 or PD-1)-specific antibodies (e.g., nivolumab and pembrolizumab).

Thus, PD1 pathway blockade has become a major focus in anticancer drug development beyond melanoma. In addition to benefiting patients with renal cell carcinoma, it has reported benefit in patients with tumors previously not considered sensitive to immunotherapies, including non-small cell lung cancer. However, there are still limitations due to toxicity associated with these immunotherapies. Thus, there is a need for an immunotherapy blocking the PD1 pathway with the best balance of high efficacy and low toxicity.

Immune checkpoint inhibition in cancer therapy has been shown to be effective for the treatment of a number of different types of cancer. However, not all cancers cells respond equally. Additionally, toxicity and the development of resistance to individual checkpoint inhibitors are problematic (Pardoll, 2012; Topalian et al., 2015). Improvements for immune checkpoint inhibitors are needed to combat aforementioned drawbacks.

Another promising therapeutic approach for cancer therapy is the use of oncolytic viruses. Treatment with oncolytic viruses by themselves and combined with other therapies elicit direct tumor cytotoxicity and potentiate activation of immune cells against tumor cells. Oncolytic viruses possess novelty in that they can also be ‘armed’ to express proteins to make them more effective (Kaufman et al., 2015).

Recent work has experimentally shown the efficacy of combining oncolytic virus with immune checkpoint inhibitor by engineering a myxoma virus to express a human extracellular portion of the PD1 checkpoint molecule (Bartee et al., 2017). PD1 is a membrane protein on T-cells that binds to PDL1 on tumor cells. This interaction triggers signaling through PD1 leading to inhibition of activation of T-cells toward tumor cells, thus protecting tumor cells from immune cell elimination (Pardoll, 2012). Upon infection of tumor cells, through direct injection in the tumor, of the myxoma virus expressing the extracellular PD1 protein (vPD1), the interaction of PDL1 on tumor and PD1 on T-cells in inhibited locally. This occurs by the extracellular PD1 protein directly binding to tumor cell PDL1 blocking T-cell PD1 from binding PDL1, leading to T-cell immune activation and anti-tumor effect.

Another major inhibitory pathway present in tumor microenvironments is the TIM3 checkpoint in which various TIM3 ligands (including GALS, phosphatidyl serine, and HMGB1) expressed by tumor cells binds to TIM3 on anti-tumor T or NK cells resulting in immune cell exhaustion. Current methods to overcome this pathway including systemic injection of antibodies which block the TIM3-GALS interaction; however, these systemic treatments are costly, time consuming, and associated with low response rates and noticeable toxicities. Thus, there is an unmet need for improved methods of inhibiting immune checkpoints.

### SUMMARY

Certain embodiments of the present disclosure provide methods and compositions comprising a recombinant oncolytic virus comprising one or more expression cassettes encoding (a) a soluble form of programmed cell death protein 1 (PD1 or PD-1), and optionally (b) interleukin 2 (IL-2) or interleukin 12 (IL-12) or interleukin 15 (IL-15), wherein the virus is replication competent. In some aspects, the soluble PD1 comprises an extracellular region of human PD1. In particular aspects, the extracellular region of human PD1 comprises SEQ ID NO: 4. In some aspects, the extracellular region of human PD1 comprises a sequence at least 80%, 85%, 90%, 95%, 96%, 97%, 98%, 99%, or 100% identical to SEQ ID NO: 4. In particular aspects, human IL-12 sequence comprises SEQ ID NOs: 7 and 8. In some aspects, the human IL-12 comprises a sequence at least 80%, 85%, 90%, 95%, 96%, 97%, 98%, 99%, or 100% identical to SEQ ID NOs: 7 and 8. In particular aspects, human IL-2 sequence comprises SEQ ID NO: 6. In some aspects, the human IL-2 comprises a sequence at least 80%, 85%, 90%, 95%, 96%, 97%, 98%, 99%, or 100% identical to SEQ ID NO: 6. In certain other aspects, a mutated or mutant version of soluble PD1 is provided to enhance the effects of combination therapies with anti-PD1 antibodies by reducing the inhibitory binding of the anti-PD1 antibody to the soluble PD1. In particular aspects, the mutant human soluble PD1 sequence comprises SEQ ID NO: 5. In some aspects, the extracellular region of human PD1 comprises a sequence at least 80%, 85%, 90%, 95%, 96%, 97%, 98%, 99%, or 100% identical to SEQ ID NO: 5.

Certain embodiments of the present disclosure provide methods and compositions comprising a recombinant oncolytic virus comprising an expression cassette encoding a mutated soluble form of PD1, wherein the virus is replication competent. In some aspects, the mutated soluble PD1 comprises an extracellular region of human PD1. In particular aspects, the extracellular region of human PD1 comprises SEQ ID NO: 5. In some aspects, the extracellular region of human PD1 comprises a sequence at least 80%, 85%, 90%, 95%, 96%, 97%, 98%, 99%, or 100% identical to SEQ ID NO: 5.

In certain aspects, IL-12 is fused to a transmembrane domain. For example, in some aspects, IL-12 can be fused to a transmembrane domain of CD28, CD8a or CD137. In further aspect, the IL-12 can be fused to a polypeptide that binds to a membrane anchored protein. Transmembrane proteins for fusions with IL-12 and methods for the same are provided, for example, in Cheng et al. 2008 (incorporated herein by reference). In some specific aspects, IL-12 is fused to a transmembrane domain that is encoded by SEQ ID NO:12. In some aspects, the oncolytic virus is encoded by a sequence at least 80%, 85%, 90%, 95%, 96%, 97%, 98%, 99%, or 100% identical to SEQ ID NO:13.

Further embodiments of the present disclosure provide a recombinant oncolytic virus comprising one or more expression cassettes encoding a soluble form of T-cell immunoglobulin and mucin-domain containing-3 (TIM3). In certain aspects, the soluble TIM3 comprises an extracellular region of murine or human TIM3.

In still a further embodiment there is provided a method of treating a disease in a subject in need thereof comprising (a) testing the subject for overexpression of GAL9; and (b) administering to a subject with increased expression of GAL9 a therapeutically effective amount of the oncolytic virus or a recombinant oncolytic virus comprising one or more expression cassettes encoding a soluble form of TIM3. In certain aspects, the subject has a cancer that exhibits increased GAL9 expression.

In certain aspects, the one or more expression cassettes may be under the control of a viral promoter. In some aspects, the viral promoter is synthetic early/late poxvirus promoter. For example, in some aspects, the synthetic early/late poxvirus promoter is about 80%, 85%, 90%, 95% or 100% identical to a sequence of SEQ ID NO: 20 (AAAATTGAAATTTTATTTTTTTTTTTTGGAATATAAATA).

In some aspects, the virus is selected from the group consisting of myxoma virus, reovirus, herpes simplex virus, Newcastle Disease virus, measles virus, retrovirus, poxvirus, rhabdovirus, picornavirus, coxsackievirus and parvovirus. In particular aspects, the oncolytic virus is myxoma virus. In some aspects, the soluble form of TIM3 or the extracellular region of PD1, such as mutated PD1, and/or IL-12, or IL-2 expression cassette(s) is/are incorporated into the myxoma genome at the viral M153R open reading frame. For example, in some aspects, an extracellular IL-12 expression construct is inserted in place of the viral M153R. In further aspects, a PD1 (extracellular domain) expression construct is inserted after viral M135 or between the viral M135 and M136. In further aspects, expression constructs for both PD1 and IL-12 can be inserted in place of the viral M153R or between the viral M135 and M136.

In a further embodiment, there is provided a pharmaceutical composition of the oncolytic virus provided herein, said pharmaceutical composition comprising a recombinant oncolytic virus comprising one or more expression cassettes encoding a soluble form of PD1, such as mutated PD1, (e.g., a recombinant oncolytic virus of the above embodiments) and optionally IL-12 or IL-2. In further aspects, the recombinant oncolytic virus comprises one or more expression cassettes encoding a soluble form of TIM3.

In another embodiment, there is provided a method of treating a disease in a subject in need thereof comprising administering an effective amount of the pharmaceutical composition provided herein, said pharmaceutical composition comprising a recombinant oncolytic virus comprising one or more expression cassettes encoding a soluble form of TIM3 or a soluble form of PD1, such as mutated PD1, and optionally IL-12 or IL-2.

In some aspects, the disease is cancer, such as a metastatic cancer. In certain aspects, the cancer has increased expression of programmed death-ligand 1 (PDL1). Thus, in some aspects, a subject for treatment according to the embodiments is a subject who has been determined to have a cancer that expresses PDL1. In other aspects, the cancer does not have increased expression of PDL1. For example, the cancer can be a melanoma, kidney cancer, colorectal cancer, breast cancer, lung cancer, head and neck cancer, brain cancer, leukemia, prostate cancer, bladder cancer, and ovarian cancer. In particular, the cancer is melanoma. In some aspects, the melanoma is metastatic melanoma. In some aspects, the cancer has metastasized to the lungs.

In certain aspects, the oncolytic virus is administered intra-arterially, intravenously, intraperitoneally, or intratumorally. In some aspects, the oncolytic virus is administered two or more times.

In some aspects, administration of the oncolytic virus results in the expression of soluble form TIM3 or soluble PD1 (e.g. mutated PD1), such as a protein of about 20-40 kDa, such as about 30 kDa. In particular aspects, the expressed, soluble PD1 (e.g. mutated PD1) is glycosylated. In certain aspects, expression of the soluble PD1 (e.g. mutated PD1) persists long-term after administration, such as for at least 3-5 days, particularly at least 6-14 days after administration. In particular aspects, expression of soluble PD1 is essentially localized to a tumor in the subject being treated.

In particular aspects, administration of the oncolytic virus does not result in alopecia, or results in at most a minor level of alopecia.

In some aspects, the method of treatment further comprises administering at least a second anti-cancer therapy to the subject. In some aspects, the second anti-cancer therapy is administered concurrently or sequentially with the recombinant virus. For example, the second anti-cancer therapy may be an immunomodulator. In other aspects, the second anti-cancer therapy is selected from chemotherapy, immunotherapy, radiotherapy, gene therapy, surgery, hormonal therapy, anti-angiogenic therapy and cytokine therapy. In some aspects, the second anti-cancer therapy comprises administration of T cells, such as CD8⁺ T cells (e.g., CD25⁺/CD69^{hi}CD8⁺ T cells). In some aspects, the immunotherapy is immune checkpoint inhibitor therapy. In specific aspects, the immune checkpoint inhibitor therapy comprises treatment with an antibody directed to PD1, PDL1, or CTLA4. In specific aspects, the antibody is Pembrolizumab, Nivolumab, Atezolizumab, Avelumab, Durvalumab, or Ipilimumab. In a preferred aspect, the treatment method comprises treatment with an antibody directed to PD1. In a particularly preferred aspect, the treatment comprises treatment with Pembrolizumab.

In another embodiment, the present disclosure provides a method of treating a disease in a subject in need thereof comprising (a) testing the subject for overexpression of PDL1; and (b) administering to a subject with increased expression of PDL1 a therapeutically effective amount of the oncolytic virus provided herein comprising a recombinant oncolytic virus comprising an expression cassette encoding soluble PD1 (e.g. mutated PD1).

In some aspects, the disease is cancer, such as a metastatic cancer. In certain aspects, the cancer has increased expression of programmed death-ligand 1 (PDL1). In other aspects, the cancer does not have increased expression of PDL1. For example, the cancer is melanoma, kidney cancer, colorectal cancer, breast cancer, lung cancer, head and neck cancer, brain cancer, leukemia, prostate cancer, bladder cancer, and ovarian cancer. In particular, the cancer is melanoma. In some aspects, the melanoma is metastatic melanoma. In some aspects, the cancer has metastasized to the lungs.

In certain aspects, the oncolytic virus is administered intra-arterially, intravenously, intraperitoneally, or intratumorally. In some aspects, the oncolytic virus is administered two or more times.

In some aspects, administration of the oncolytic virus results in the expression of soluble form of TIM3 or soluble PD1, such as mutant PD1, such as a protein of about 20-40 kDa, such as about 30 kDa. In particular aspects, the expressed, soluble PD1 is glycosylated. In certain aspects, expression of the soluble TIM3 or soluble PD1 persists long-term after administration, such as for at least 3-5 days, particularly at least 6-14 days after administration. In particular aspects, expression of soluble TIM3 or PD1 is essentially localized to a tumor in the subject being treated. In particular aspects, administration of the oncolytic virus does not result in alopecia, or results in at most a minor level of alopecia.

In some aspects, the method of treatment further comprises administering at least a second anti-cancer therapy to the subject. In some aspects, the second anti-cancer therapy is administered concurrently or sequentially with the recombinant virus. For example, the second anti-cancer therapy may be an immunomodulator. In other aspects, the second anti-cancer therapy is chemotherapy, immunotherapy, radiotherapy, gene therapy, surgery, hormonal therapy, anti-angiogenic therapy or cytokine therapy. In some aspects, the second anti-cancer therapy comprises administration of T cells, such as CD8⁺ T cells (e.g., CD25⁺/CD69^{hi}CD8⁺ T cells). 40. In some aspects, the immunotherapy is immune checkpoint inhibitor therapy. In specific aspects, the immune checkpoint inhibitor therapy comprises treatment with an antibody directed to PD1, PDL1, or CTLA4. In specific aspects, the antibody is Pembrolizumab, Nivolumab, Atezolizumab, Avelumab, Durvalumab, or Ipilimumab.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%. Most preferred is a composition in which no amount of the specified component can be detected with standard analytical methods.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

Other objects, features and advantages of the present disclosure will become apparent from the following detailed description. It should be understood, however, that the detailed description and the specific examples, while indicating preferred embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIG. 1—Schematics of recombinant viral genomic structures.

FIG. 2—vPD1-IL2 efficacy study in subcutaneous B16F10 (B16F10 PD1L-KO) contralateral xenograft model.

FIG. 3—vPD1-IL12 efficacy study in subcutaneous B16F10 (B16F10 PD1L-KO) contralateral xenograft model.

FIG. 4—vPD1-IL15 efficacy study in subcutaneous B16F10 (B16F10 PD1L-KO) contralateral xenograft model.

FIG. 5—vPD1-IL18 efficacy study in subcutaneous B16F10 (B16F10 PD1L-KO) contralateral xenograft model.

FIG. 6—In vivo SC contralateral model. Starting tumor size.

FIG. 7—In vivo SC contralateral model. Treatment results are shown. The studies demonstrate that vPD1/IL12 constructs were superior to other constructs tested.

FIG. 8—Sequence alignment of the C′D loop in ectodomains of PD-1. Secondary structural elements of human PD-1 (hPD-1) are shown on top of the alignment while those of murine PD-1 (mPD-1) are shown at the bottom

FIG. 9—Schematic depicting therapy with soluble TIM3 myxoma virus.

FIG. 10: MYXV therapy induces TIM3 on CD8+ T cells and NK cells. Analysis of TIM3 expression on the indicated immunological subsets six days after initiation of viral treatment.

FIGS. 11A-11C: TIM3 blockade improves MYXV treatment of melanoma. SQ B16/F10 tumors were treated as indicated. (A) Tumor volume as a percent of starting volume. Complete eradication of visible tumor is marked with white circles. (B) Overall survival of animals. (C) Example of alopecia observed in animals.

FIGS. 12A-12D: vTIM3 secretes soluble TIM3 from infected cells. (A) Schematic of the genomic structure of vGFP and vTIM3. (B) Production of new virus in B16/F10 cells. (C) MTT assay analyzing cellular viability 24 hours post infection. (D) Expression of TIM3 transgene.

FIGS. 13A-13C: vTIM3 duplicates efficacy of combination therapy with reduced toxicities. (A) Tumor volume as a percent of starting volume. Complete eradication of visible tumor is marked with white circles. (B) Overall survival of animals. (C) Average alopecia score observed in animals treated with the indicated therapy.

FIGS. 14A-14B: Generation of mutations in TIM3. (A) Schematic of proposed mutations for TIM3 transgenes. (B) Expression of mutated TIM3 out of newly generated recombinant vTIM3 mutant viruses. Note that the GALS mutant runs at a lower MW due to the loss of glycosylation.

FIGS. 15A-15C: (A) Schematic depicting mouse study. (B) Individual tumor growth over time. (C) Overall survival.

FIGS. 16A-B: vPD1 is effective against localized but not metastatic tumors. Single (A) or contralateral (B) B16/F10 tumors were established in syngeneic mice. Tumors on the left flank were then treated with either control virus (vGFP) or vPD1. Tumors on the right flank in contralateral model were left untreated. Responsiveness of individual tumors and overall survival were then monitored.

FIGS. 17A-17D: MYXV expressing both soluble PD1 and IL12 is highly effective against metastatic disease. (A) Genomic structure of viruses expressing both soluble PD1 and proinflammatory cytokines. Contralateral LLC tumors were established in syngeneic mice. Tumors on the left flank were then treated as indicated and tumors on the right flank were left untreated. (B) Responsiveness of individual tumors and (C) overall survival were then monitored. (D) Picture of mouse bearing bulky, contralateral LLC tumors treated as above.

FIGS. 18A-18C: vPD1/IL12 is effective against metastatic lung cancer. (A) Contralateral LLC tumors were established in syngeneic mice. Tumors on the left flank were then treated as indicated and tumors on the right flank were left untreated. (B) Responsiveness of individual tumors and (C) overall survival were then monitored.

FIGS. 19A-19C: vPD1/IL12 is effective against metastatic melanoma. (A) Contralateral B16/F10 tumors were established in syngeneic mice. Tumors on the left flank were then treated as indicated and tumors on the right flank were left untreated. (B) Responsiveness of individual tumors and (C) overall survival were then monitored.

FIGS. 20A-20C: vPD1/IL12 is effective against spontaneously metastatic breast cancer. (A) Single 4T1 tumors were established in syngeneic mice and allowed to establish and metastasize. Primary tumors were then treated as indicated. (B) Responsiveness of individual tumors and (C) overall survival were then monitored.

### DESCRIPTION OF ILLUSTRATIVE EMBODIMENTS

Two of the major inhibitory pathways present in tumor microenvironments are the PD1-PDL1 checkpoint in which PDL1 expressed on tumor cells binds to PD1 on anti-tumor T cells resulting in T cell exhaustion as well as the TIM3 checkpoint. Current methods to overcome these pathways include systemic injection of antibodies which block the PD1-PDL1 or TIM3-GAL9 interaction; however, these systemic treatments are costly, time consuming and associated with low response rates and noticeable toxicities.

Certain embodiments of the present disclosure provide compositions and methods for targeting the PD1-PDL1 or TIM3 pathway in cancer. In some aspects, a recombinant oncolytic virus is provided, which has been engineered to express the extracellular portion of the human PD1 protein or TIM3 protein along with IL-2 or IL-12. In certain aspects, the oncolytic virus is a replication competent virus such as myxoma virus. In particular, the extracellular region of PD1 or TIM3 and IL-2 or IL-12 can be encoded by one or more expression cassettes that is integrated into a region of the viral genome that is not necessary for replication. In the present studies, the oncolytic virus provided tumor inhibition that can significantly improve outcomes during oncolytic virotherapy.

Accordingly, further embodiments of the present disclosure provide methods of cancer treatment comprising administering the recombinant oncolytic virus expressing the soluble form of PD1 or TIM3 and IL-2 or IL-12 are also provided. Thus, the present aspects of the disclosure provide methods and compositions for a therapy targeting the PD1-PDL1 or TIM3-GAL9 pathway, in combination with cytokine therapy, with a low toxicity and high response rate.

### I. DEFINITIONS

The term “oncolytic virus,” as used herein, refers to a virus capable of selectively replicating in and slowing the growth or inducing the death of a cancerous or hyperproliferative cell, either in vitro or in vivo, while having no or minimal effect on normal cells. Exemplary oncolytic viruses include vesicular stomatitis virus (VSV), Newcastle disease virus (NDV), herpes simplex virus (HSV), reovirus, measles virus, retrovirus, influenza virus, Sinbis virus, vaccinia virus, and adenovirus.

A “promoter” is a control sequence that is a region of a nucleic acid sequence at which initiation and rate of transcription are controlled. It may contain genetic elements at which regulatory proteins and molecules may bind, such as RNA polymerase and other transcription factors, to initiate the specific transcription of a nucleic acid sequence. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence.

The term “innate immunity” or “innate immune response” refers to the repertoire of host defenses, both immunological and nonimmunological, that exist prior to or independent of exposure to specific environmental antigens, such as a microorganism or macromolecule, etc. For example, the first host immune response to an antigen involves the innate immune system.

The term “immunogen” or “antigen,” as used herein, refers to an agent that is recognized by the immune system when introduced into a subject and is capable of eliciting an immune response. In certain embodiments, the immune response generated is an innate cellular immune response and the recombinant oncolytic viruses of the instant disclosure are capable of suppressing or reducing the innate cellular immune response.

As employed herein, the phrase “an effective amount,” refers to a dose sufficient to provide concentrations high enough to impart a beneficial effect on the recipient thereof. The specific therapeutically effective dose level for any particular subject will depend upon a variety of factors including the disorder being treated, the severity of the disorder, the activity of the specific compound, the route of administration, the rate of clearance of the compound, the duration of treatment, the drugs used in combination or coincident with the compound, the age, body weight, sex, diet, and general health of the subject, and like factors well known in the medical arts and sciences.

As used herein the term “multiplicity of infection” (MOI) means the number of infectious virus particles added per cell.

### II. ONCOLYTIC VIRUSES

### A. Oncolytic Viral Platforms

In one aspect, the present disclosure generally pertains to recombinant, replication competent, oncolytic viruses. In one embodiment, there is provided a recombinant oncolytic virus having a heterologous nucleic acid sequence encoding PD1 or TIM3 Oncolytic viruses that can be administered according to the methods of the disclosure include, without limitation, adenoviruses (e.g. Delta-24, Delta-24-RGD, ICOVIR-5, ICOVIR-7, Onyx-015, ColoAd1, H101, AD5/3-D24-GMCSF), reoviruses, herpes simplex virus (HSV; OncoVEX GMCSF), Newcastle Disease virus, measles viruses, retroviruses (e.g. influenza viruses), poxviruses (e.g. vaccinia virus including Copenhagen, Western Reserve, Wyeth strains), myxoma viruses, rhabdoviruses (e.g vesicular stomatitis virus (VSV)), picornaviruses (e.g. Seneca Valley virus; SVV-001), coxsackievirus and parvovirus.

In one embodiment, the recombinant oncolytic virus comprises myxoma virus. Myxoma virus (MYVX) is a member of the Poxviridae family and prototype for the genus Leporipoxvirus. It is pathogenic only for European rabbits (Oryctolagus cuniculus), in which it causes a lethal disease called myxomatosis, and for two North American species, Sylvilagus audubonni and Sylvilagus nuttalli, in which it causes a less severe disease. Myxoma virus replicates exclusively in the cytoplasm of the host cell, and its genome encodes 171 open reading frames (Smallwood et al., 2010). A number of these genes encode proteins that can interfere with or modulate host defense mechanisms, and several show promise in a clinical setting.

Like other members of the poxvirus family, the myxoma virus genome consists of a single double stranded DNA (dsDNA), the central part of the which encodes approximately 100 essential genes that are conserved among the members of poxvirus genera. The rest of the genes, including two copies each of the 12 genes that map within the terminal inverted repeats, encode proteins that interfere with and modulate host defense mechanisms. A number of these proteins share a sequence similarity with host cellular genes, suggesting a coevolutionary path (Johnston and McFadden, 2003). Some, called viroceptors, are secreted and able to bind specific ligands such as TNF, for example. Others, known as virokines, are also secreted, and imitate host immune inhibitors, while viromitigators function as host range factors that inhibit apoptosis (Johnston and McFadden, 2003; Kerr and McFadden, 2002). These characteristics give myxoma virus possible utility in a number of therapeutic settings. One of the myxoma virus-encoded immunomodulatory proteins, Serp-1, is in clinical trials for acute unstable coronary syndromes (e.g., unstable angina and small heart attacks). The M-T7 protein of myxoma virus, a secreted glycoprotein that inhibits rabbit gamma interferon, has also been shown to inhibit inflammatory responses in rabbit models of balloon angioplasty injury to arteries (Liu et al., 2000), and it is likely that a variety of other immunomodulatory proteins can be developed as anti-inflammatory or anti-immune therapeutics.

Myxoma virus has been shown to productively infect a variety of human cancer cell lines originated from a diverse group of tissues (Sypula et al., 2004), and therefore has the potential for development as an oncolytic virus useful in treatment against a variety of cancers. Wildtype myxoma virus can selectively infect and kill cells, including human cells, which have a deficient innate anti-viral response, for example, cells that are non-responsive to interferon, as described in the application PCT/CA2004/000341, which is herein fully incorporated by reference. Furthermore, myxoma virus is adept at evading and interfering with the host immune response and might serve as a source of immunomodulatory proteins that can be used as therapeutic agents in a variety of clinical settings (Lucas and McFadden, 2004). Additionally, although myxoma virus is not infectious in humans, it is able to productively infect a number of human cancer cell lines, but not normal human cells, and has also been shown to increase survival time in mouse models of human glioma. These characteristics suggest that myxoma virus could prove to be a viable therapeutic agent in a variety of clinical settings, including as an anti-inflammatory or anti-immune therapy, or as an oncolytic agent.

Myxoma virus has established oncolytic potential against a variety of malignancies including myeloma, melanoma, glioblastoma, pancreatic cancer, and others. The virus is thought to exhibit anti-tumor effects through two distinct mechanisms. First, the virus directly infects and kills tumor cells. Second, viral infection of tumor cells induces a secondary anti-tumor immune response. While the combination of these mechanisms is effective at debulking primary tumors, it often fails to produce long-term cures due to immune inhibition within the tumor microenvironment.

The myxoma virus of the present disclosure can be attenuated to enhance anti-tumor activity. For example, the myxoma virus can be genetically modified to inactivate one or more genes. In particular, myxoma virus that does not express functional M135R is useful for treatment of cells having a deficient innate anti-viral response, including for oncolytic studies, since this virus provides a safer alternative for oncolytic viral therapy as no unusual containment strategies should be needed for patients undergoing treatment (U.S. Patent Application No. 20090035276, incorporated herein by reference). In certain aspects, the myxoma virus is an attenuated strain of myxoma virus such as the SG33 strain (U.S. Pat. No. 8,613,915, incorporated herein by reference). An attenuated myxoma virus which can be used in accordance with the disclosure may be obtained from a virulent wild-type myxoma virus, especially by deletion of one or more of the genes M151R, M152R, M153R, M154L, M156R, and M001R, and preferably by the additional deletion of one or more of the genes M008.1R, M008R, M007R, M006R, M005R, M004.1R, M004R, M003.2R, M003.1R, and M002R.

Myxoma virus can be propagated in a number of cell lines, including adherent cells and suspension cultures, and minimal purification is required. For example, myxoma virus can grow in several cell lines, including RK13 (rabbit kidney epithelial), BHK-21 (baby hamster kidney), BGMK (Buffalo green monkey kidney), Vero (African green monkey kidney epithelial), BSC-40 (African green monkey kidney), and CV-1 (African green monkey kidney fibroblast) cells. Minimal purification is needed to provide a stock that is appropriate for both in vitro and in vivo work. Protocols for propagating, purifying, and quantifying stocks of myxoma virus are known in the art (Smallwood et al., 2010, incorporated herein by reference).

### B. Recombinant Oncolytic Viruses

The recombinant virus can be constructed by procedures known in the art to generate recombinant viruses. An expression cassette encoding PD1, such as mutant PD1, or TIM3 is inserted into the genome of an oncolytic virus at a region nonessential for viral replication. For example, the expression cassette can be integrated in myxoma virus at an intergenic region, such as between the M135 and M136 open reading frames. The recombinant virus can comprise an expression cassette comprising a nucleotide sequence which is at least about 50%, 55%, 60%, 65%, 70%, 75%, 80%, 85%, 90%, 91%, 92%, 93%, 94%, 95%, 96%, 97%, 98%, 99% or more identical to the nucleotide sequence (e.g., to the entire length of the nucleotide sequence) of the extracellular portion of human PD1, which is shown in SEQ ID NO:3. The nucleotide sequence of SEQ ID NO:3 can be optimized for expression in the recombinant virus, for example, through codon optimization. The expression cassette can encode for soluble TIM3 (SEQ ID NO:11) or sequence with at least 50%, 55%, 60%, 65%, 70%, 75%, 80%, 85%, 90%, 91%, 92%, 93%, 94%, 95%, 96%, 97%, 98%, 99% or more identical to SEQ ID ID NO:11).

Homologous recombination (HR), also known as general recombination, is a type of genetic recombination used in all forms of life in which nucleotide sequences are exchanged between two similar or identical strands of DNA. The technique has been the standard method for genome engineering in mammalian cells since the mid-1980s. The process involves several steps of physical breaking and the eventual rejoining of DNA. This process is most widely used to repair potentially lethal double-strand breaks in DNA. In addition, homologous recombination produces new combinations of DNA sequences during meiosis, the process by which eukaryotes make germ cells like sperm and ova. These new combinations of DNA represent genetic variation in offspring which allow populations to evolutionarily adapt to changing environmental conditions over time. Homologous recombination is also used in horizontal gene transfer to exchange genetic material between different strains and species of bacteria and viruses. Homologous recombination is also used as a technique in molecular biology for introducing genetic changes into target organisms.

Expression cassettes included in vectors useful in the disclosure preferably contain (in a 5′-to-3′ direction) a eukaryotic transcriptional promoter operably linked to a protein-coding sequence. Non-limiting examples of promoters include early or late viral promoters, such as, SV40 early or late promoters, cytomegalovirus (CMV) immediate early promoters, Rous Sarcoma Virus (RSV) early promoters; eukaryotic cell promoters, such as, e.g., beta actin promoter (Ng, 1989; Quitsche et al., 1989), GADPH promoter (Alexander et al., 1988, Ercolani et al., 1988), metallothionein promoter (Karin et al., 1989; Richards et al., 1984); and concatenated response element promoters, such as cyclic AMP response element promoters (cre), serum response element promoter (sre), phorbol ester promoter (TPA) and response element promoters (tre) near a minimal TATA box. It is also possible to use human growth hormone promoter sequences (e.g., the human growth hormone minimal promoter described at Genbank, accession no. X05244, nucleotide 283-341) or a mouse mammary tumor promoter (available from the ATCC, Cat. No. ATCC 45007). A specific example could be a synthetic early/late (sE/L) poxvirus promoter (see, e.g., the promoter of the construct to SEQ ID NO: 10).

The expression cassette is introduced to cells which are then infected with the unmodified oncolytic virus to produce the recombinant virus. Introduction of the expression cassette into cells may use any suitable methods for nucleic acid delivery for transformation of a cell, as described herein or as would be known to one of ordinary skill in the art. Such methods include, but are not limited to, direct delivery of DNA such as by ex vivo transfection (Wilson et al., 1989, Nabel et al, 1989), by injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harland and Weintraub, 1985; U.S. Pat. No. 5,789,215, incorporated herein by reference); by electroporation (U.S. Pat. No. 5,384,253, incorporated herein by reference; Tur-Kaspa et al., 1986; Potter et al., 1984); by calcium phosphate precipitation (Graham and Van Der Eb, 1973; Chen and Okayama, 1987; Rippe et al., 1990); by using DEAE-dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991) and receptor-mediated transfection (Wu and Wu, 1987; Wu and Wu, 1988); by microprojectile bombardment (PCT Application Nos. WO 94/09699 and 95/06128; U.S. Pat. Nos. 5,610,042; 5,322,783 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); by Agrobacterium-mediated transformation (U.S. Pat. Nos. 5,591,616 and 5,563,055, each incorporated herein by reference); by desiccation/inhibition-mediated DNA uptake (Potrykus et al., 1985), and any combination of such methods. Through the application of techniques such as these, organelle(s), cell(s), tissue(s) or organism(s) may be stably or transiently transformed.

The recombinant virus is then purified from the cells such as by a selectable marker. Such markers would confer an identifiable change to the cell permitting easy identification of cells containing the expression vector. Generally, a selection marker is one that confers a property that allows for selection. A positive selection marker is one in which the presence of the marker allows for its selection, while a negative selection marker is one in which its presence prevents its selection. An example of a positive selection marker is a drug resistance marker. Usually the inclusion of a drug selection marker aids in the cloning and identification of transformants, for example, genes that confer resistance to neomycin, puromycin, hygromycin, DHFR, GPT, zeocin and histidinol are useful selection markers. In addition to markers conferring a phenotype that allows for the discrimination of transformants based on the implementation of conditions, other types of markers including screenable markers such as GFP, whose basis is colorimetric analysis, are also contemplated. Alternatively, screenable enzymes as negative selection markers such as herpes simplex virus thymidine kinase (tk) or chloramphenicol acetyltransferase (CAT) may be utilized. The marker used is not believed to be important, so long as it is capable of being expressed simultaneously with the nucleic acid encoding a gene product. For example, the recombinant oncolytic virus can be untagged or express fluorescent proteins such as green fluorescent protein (GFP), red fluorescent protein (RFP), tomato Red (tdRed), or other fluorescent proteins. Further examples of selection and screenable markers are well known to one of skill in the art.

The transgene expressing tomato Red fluorescent (tdTr), which serves as a fluorescent marker for myxoma replication in vitro and in vivo, has been described in Liu et al. (2009) J. Virology 83:5933-5938. Liu observed that a myxovirus expressing IL-15 fused to tdTr (vMyx-IL-15-tdTr) was significantly attenuated and failed to induce lethal myxomatosis in rabbits. The construct secreted IL-15 and supported normal virus replication. Thus, Liu concluded that vMyx-IL-15-tdTr was a safe candidate for in vivo animal studies of oncolytic virotherapy, and tdTr is a suitable marker for use in recombinant myxovirus.

If desired, one or more genetic elements, such as transgenes expressing fluorescent markers, can be excised from a viral transposon, using methods known in the art, such as Flp recombinase or Cre-lox recombination-based systems.

### C. PD1

Programmed cell death protein 1, also known as PD-1 and CD279 (cluster of differentiation 279), is a protein found on the surface of cells that has a role in regulating the immune system's response to the cells of the human body by down-regulating the immune system and promoting self-tolerance by suppressing T cell inflammatory activity. This prevents autoimmune diseases, but it can also prevent the immune system from killing cancer cells.

The amino acid sequence of the extracellular domain of human PD-1 is found at Uniprot Accession Number Q15116, SEQ ID NO: 4, and is 168 amino acids in length, which includes a 20 amino acid signal sequence which may be replaced by a different signal sequence, or omitted from the PD-1 sequences of the present disclosure, when not needed in order to direct secretion.

PD-1 is an immune checkpoint and guards against autoimmunity through two mechanisms. First, it promotes apoptosis (programmed cell death) of antigen-specific T-cells in lymph nodes. Second, it reduces apoptosis in regulatory T cells (anti-inflammatory, suppressive T cells). PD-1 inhibitors, a new class of drugs that block PD-1, activate the immune system to attack tumors and are used to treat certain types of cancer.

The PD-1 protein in humans is encoded by the PDCD1 gene. PD-1 is a cell surface receptor that belongs to the immunoglobulin superfamily and is expressed on T cells and pro-B cells. PD-1 binds two ligands, PD-L1 and PD-L2. PD-1 is a type I membrane protein of 268 amino acids. PD-1 is a member of the extended CD28/CTLA-4 family of T cell regulators. The protein's structure includes an extracellular IgV domain followed by a transmembrane region and an intracellular tail. The intracellular tail contains two phosphorylation sites located in an immunoreceptor tyrosine-based inhibitory motif and an immunoreceptor tyrosine-based switch motif, which suggests that PD-1 negatively regulates T-cell receptor TCR signals. This is consistent with binding of SHP-1 and SHP-2 phosphatases to the cytoplasmic tail of PD-1 upon ligand binding. In addition, PD-1 ligation up-regulates E3-ubiquitin ligases CBL-b and c-CBL that trigger T cell receptor down-modulation. PD-1 is expressed on the surface of activated T cells, B cells, and macrophages, suggesting that compared to CTLA-4, PD-1 more broadly negatively regulates immune responses.

PD-1 has two ligands, PD-L1 and PD-L2, which are members of the B7 family. PD-L1 protein is upregulated on macrophages and dendritic cells (DC) in response to LPS and GM-CSF treatment, and on T cells and B cells upon TCR and B cell receptor signaling, whereas in resting mice, PD-L1 mRNA can be detected in the heart, lung, thymus, spleen, and kidney. PD-L1 is expressed on almost all murine tumor cell lines, including PA1 myeloma, P815 mastocytoma, and B16 melanoma upon treatment with IFN-γ. PD-L2 expression is more restricted and is expressed mainly by DCs and a few tumor lines.

Several lines of evidence suggest that PD-1 and its ligands negatively regulate immune responses. PD-1 knockout mice have been shown to develop lupus-like glomerulonephritis and dilated cardiomyopathy on the C57BL/6 and BALB/c backgrounds, respectively. In vitro, treatment of anti-CD3 stimulated T cells with PD-L1-Ig results in reduced T cell proliferation and IFN-γ secretion. IFN-γ is a key pro-inflammatory cytokine that promotes T cell inflammatory activity. Reduced T cell proliferation was also correlated with attenuated IL-2 secretion and together, these data suggest that PD-1 negatively regulates T cell responses.

Experiments using PD-L1 transfected DCs and PD-1 expressing transgenic (Tg) CD4⁺ and CD8⁺ T cells suggest that CD8⁺ T cells are more susceptible to inhibition by PD-L1, although this could be dependent on the strength of TCR signaling. Consistent with a role in negatively regulating CD8⁺ T cell responses, using an LCMV viral vector model of chronic infection, Rafi Ahmed's group showed that the PD-1-PD-L1 interaction inhibits activation, expansion and acquisition of effector functions of virus specific CD8⁺ T cells, which can be reversed by blocking the PD-1-PD-L1 interaction.

Expression of PD-L1 on tumor cells inhibits anti-tumor activity through engagement of PD-1 on effector T cells. Expression of PD-L1 on tumors is correlated with reduced survival in esophageal, pancreatic and other types of cancers, highlighting this pathway as a target for immunotherapy. Triggering PD-1, expressed on monocytes and up-regulated upon monocytes activation, by its ligand PD-L1 induces IL-10 production which inhibits CD4 T-cell function.

In mice, expression of this gene is induced in the thymus when anti-CD3 antibodies are injected and large numbers of thymocytes undergo apoptosis. Mice deficient for this gene bred on a BALB/c background developed dilated cardiomyopathy and died from congestive heart failure. These studies suggest that this gene product may also be important in T cell function and contribute to the prevention of autoimmune diseases. Overexpression of PD1 on CD8+T cells is one of the indicators of T-cell exhaustion (e.g., in chronic infection or cancer).

PD-L1, the primary ligand for PD1, is highly expressed in several cancers and hence the role of PD1 in cancer immune evasion is well established. Monoclonal antibodies targeting PD-1 that boost the immune system are being developed for the treatment of cancer. Many tumor cells express PD-L1, an immunosuppressive PD-1 ligand; inhibition of the interaction between PD-1 and PD-L1 can enhance T-cell responses in vitro and mediate preclinical antitumor activity. This is known as immune checkpoint blockade.

Combination therapy using both anti-PD1 along with anti-CTLA4 therapeutics have emerged as important tumor treatments within the field of checkpoint inhibition. A combination of PD1 and CTLA4 antibodies has been shown to be more effective than either antibody alone in the treatment of a variety of cancers. The effects of the two antibodies do not appear to be redundant. Anti-CTLA4 treatment leads to an enhanced antigen specific T cell dependent immune reaction while anti-PD-1 appears to reactivate CD8+ T cells ability to lyse cancer cells.

In clinical trials, combination therapy has been shown to be effective in reducing tumor size in patients that are unresponsive to single co-inhibitory blockade, despite increasing levels of toxicity due to anti-CTLA4 treatment. A combination of PD1 and CTLA4 induced up to a ten-fold higher number of CD8+ T cells that are actively infiltrating the tumor tissue. The authors hypothesized that the higher levels of CD8+ T cell infiltration was due to anti-CTLA-4 inhibited the conversion of CD4 T cells to T regulator cells and further reduced T regulatory suppression with anti-PD-1. This combination promoted a more robust inflammatory response to the tumor that reduced the size of the cancer. Most recently, the FDA has approved a combination therapy with both anti-CTLA4 (ipilimumab) and anti-PD1 (nivolumab) in October 2015.

The molecular factors and receptors necessary making a tumor receptive to anti-PD1 treatment remains unknown. PD-L1 expression on the surface on cancer cells plays a significant role. PD-L1 positive tumors were twice as likely to respond to combination treatment. However patients with PD-L1 negative tumors also have limited response to anti-PD1, demonstrating that PD-L1 expression is not an absolute determinant of the effectiveness of therapy.

Higher mutational burden in the tumor is correlated with a greater effect of the anti-PD1 treatment. In clinical trials, patients who benefited from anti-PD1 treatment had cancers, such as melanoma, bladder cancer, and gastric cancer, that had a median higher average number of mutations than the patients who do did not respond to the therapy. However, the correlation between higher tumor burden and the clinical effectiveness of PD-1 immune blockade is still uncertain.

### D. IL-12

Interleukin 12 (IL-12) is an interleukin that is naturally produced by dendritic cells, macrophages, neutrophils, and human B-lymphoblastoid cells (NC-37) in response to antigenic stimulation. IL-12 is composed of a bundle of four alpha helices. It is a heterodimeric cytokine encoded by two separate genes, IL-12A (p35) and IL-12B (p40). The active heterodimer (referred to as ‘p70’), and a homodimer of p40 are formed following protein synthesis. The amino acid sequence of human IL-12 alpha subunit is found at Uniprot Accession Number P29459, SEQ ID NO: 7, and is 219 amino acids in length, which includes a 22 amino acid signal sequence which may be replaced by a different signal sequence, or omitted from the IL-12 alpha subunit sequences of the present invention, when not needed in order to direct secretion. The amino acid sequence of human IL-12 beta subunit is found at Uniprot Accession Number P29460, SEQ ID NO: 8, and is 328 amino acids in length, which includes a 22 amino acid signal sequence, which may be replaced by a different signal sequence, or omitted from the IL-12 beta subunit sequences of the present invention, when not needed in order to direct secretion. The nucleotide sequences encoding IL-12 alpha and beta subunits can be optimized for expression in the recombinant virus, for example, through codon optimization.

In certain embodiments, the IL-12 alpha subunit and IL-12 beta subunit may be expressed as a fusion protein from a single DNA construct. In such cases, only a single signal peptide is required, preferably at the N-terminal end of the expressed fusion protein. In such cases, a flexible linker peptide may be used to join the IL-12 alpha subunit and IL-12 beta subunits. Suitable linker peptide sequences are known in the art, and include, for example (GGGS)ₙ, where n=1 to 4.

IL-12 is involved in the differentiation of naive T cells into Th1 cells. It is known as a T cell-stimulating factor, which can stimulate the growth and function of T cells. It stimulates the production of interferon-gamma (IFN-γ) and tumor necrosis factor-alpha (TNF-α) from T cells and natural killer (NK) cells, and reduces IL-4 mediated suppression of IFN-γ. T cells that produce IL-12 have a coreceptor, CD30, which is associated with IL-12 activity.

IL-12 plays an important role in the activities of natural killer cells and T lymphocytes. IL-12 mediates enhancement of the cytotoxic activity of NK cells and CD8+ cytotoxic T lymphocytes. There also seems to be a link between IL-2 and the signal transduction of IL-12 in NK cells. IL-2 stimulates the expression of two IL-12 receptors, IL-12R-β1 and IL-12R-β2, maintaining the expression of a critical protein involved in IL-12 signaling in NK cells. Enhanced functional response is demonstrated by IFN-γ production and killing of target cells.

IL-12 also has anti-angiogenic activity, which means it can block the formation of new blood vessels. It does this by increasing production of interferon gamma, which in turn increases the production of a chemokine called inducible protein-10 (IP-10 or CXCL10). IP-10 then mediates this anti-angiogenic effect. Because of its ability to induce immune responses and its anti-angiogenic activity, there has been an interest in testing IL-12 as a possible anti-cancer drug. However, it has not been shown to have substantial activity in the tumors tested to this date. There is a link that may be useful in treatment between IL-12 and the diseases psoriasis and inflammatory bowel disease.

IL-12 binds to the IL-12 receptor, which is a heterodimeric receptor formed by IL-12Rβ1 and IL-12Rβ2. IL-12Rβ2 is considered to play a key role in IL-12 function, since it is found on activated T cells and is stimulated by cytokines that promote Th1 cells development and inhibited by those that promote Th2 cells development. Upon binding, IL-12R-β2 becomes tyrosine phosphorylated and provides binding sites for kinases, Tyk2 and Jak2. These kinases are important in activating critical transcription factor proteins such as STAT4 that are implicated in IL-12 signaling in T cells and NK cells. This pathway is known as the JAK-STAT pathway.

IL-12 is linked with autoimmunity. Administration of IL-12 to people suffering from autoimmune diseases was shown to worsen the autoimmune phenomena. This is believed to be due to its key role in induction of Th1 immune responses. In contrast, IL-12 gene knock-out in mice or a treatment of mice with IL-12 specific antibodies ameliorated the disease.

Interleukin 12 (IL-12) is produced by activated antigen-presenting cells (dendritic cells, macrophages). It promotes the development of Th1 responses and is a powerful inducer of IFNγ production by T and NK cells.

A child with Bacillus Calmette-Guérin and Salmonella enteritidis infection was found to have a large homozygous deletion within the IL-12 p40 subunit gene, precluding expression of functional IL-12 p70 cytokine by activated dendritic cells and phagocytes. As a result, IFNγ production by the child's lymphocytes was markedly impaired. This suggested that IL-12 is essential for protective immunity to intracellular bacteria such as mycobacteria and Salmonella.

Support is lent to this idea by the observation that a receptor for IL-12 is important for IFNγ production by lymphocytes. T and NK cells from seven unrelated patients who had severe idiopathic mycobacterial and Salmonella infections failed to produce IFNγ when stimulated with IL-12. The patients were otherwise healthy. They were found to have mutations in the IL-12 receptor (31 chain, resulting in premature stop codons in the extracellular domain, resulting in unresponsiveness to this cytokine, again demonstrating IL-12's crucial role in host defense.

Defective Th1 and Th17 immune responses leading to chronic mucocutaneous candidiasis result from a mutation further downstream in the IL-12 signaling pathway. The trait was mapped to mutations in the STAT1 gene, which were associated with lower production of interferon-γ, IL-17, and IL-22 in response to IL-12 or IL-23 receptor associated Jak2 and Tyk2 activity.

### E. IL-2

Interleukin-2 (IL-2) is an interleukin, a type of cytokine signaling molecule in the immune system. It is a protein that regulates the activities of white blood cells (leukocytes, often lymphocytes) that are responsible for immunity. IL-2 is part of the body's natural response to microbial infection, and in discriminating between foreign (“non-self”) and “self”. IL-2 mediates its effects by binding to IL-2 receptors, which are expressed by lymphocytes. The amino acid sequence of human IL-2 is found at Uniprot Accession Number P60568, SEQ ID NO: 6, and is 153 amino acids in length, which includes a 20 amino acid signal sequence, which may be replaced by a different signal sequence, or omitted from the IL-2 sequences of the present invention, when not needed in order to direct secretion. The nucleotide sequence encoding IL-2 can be optimized for expression in the recombinant virus, for example, through codon optimization.

In a preferred embodiment, the IL-2 useful in the present invention is the high affinity variant IL-2 amino acid sequence of SEQ ID NO: 9, which includes a 20 amino acid signal sequence, which may be replaced by a different signal sequence, and which also contains C-terminal His tag. Levin et al. (2012) Nature 484:529-533. Either or both of the signal sequence and His tag may be omitted, if not required for function of the IL-2.

IL-2 is a member of a cytokine family, each member of which has a four alpha helix bundle; the family also includes IL-4, IL-7, IL-9, IL-15 and IL-21. IL-2 signals through the IL-2 receptor, a complex consisting of three chains, termed alpha, beta and gamma. The gamma chain is shared by all family members.

The IL-2 Receptor (IL-2R) a subunit has low affinity for its ligand but has the ability (when bound to the β and Υ subunit) to increase the IL-2R affinity 100-fold. Heterodimerization of the β and Υ subunits of IL-2R is essential for signaling in T cells.

Gene expression regulation for IL-2 can be on multiple levels or by different ways. One of the checkpoints is signaling through TCR receptor, antigen receptor of T-lymphocytes after recognizing MHC-peptide complex. Signaling pathway from TCR then goes through phospholipase-C (PLC) dependent pathway. PLC activates 3 major transcription factors and their pathways: NFAT, NFkB and AP-1. After costimulation from CD28 the optimal activation of expression of IL-2 and these pathways is induced.

At the same time Oct-1 is expressed. It helps the activation. Octl is expressed in T-lymphocytes and Oct2 is induced after cell activation. NFAT has multiple family members, all of them are located in cytoplasm and signaling goes through calcineurin, NFAT is dephosphorylated and therefore translocated to the nucleus. AP-1 is a dimer and is composed of c-Jun and c-Fos proteins. It cooperates with other transcription factors including NFkB and Oct. NFkB is translocated to the nucleus after costimulation through CD28. NFkB is a heterodimer and there are two binding sites on the IL-2 promoter.

IL-2 has essential roles in key functions of the immune system, tolerance and immunity, primarily via its direct effects on T cells. In the thymus, where T cells mature, it prevents autoimmune diseases by promoting the differentiation of certain immature T cells into regulatory T cells, which suppress other T cells that are otherwise primed to attack normal healthy cells in the body. IL-2 also promotes the differentiation of T cells into effector T cells and into memory T cells when the initial T cell is also stimulated by an antigen, thus helping the body fight off infections. Its expression and secretion is tightly regulated and functions as part of both transient positive and negative feedback loops in mounting and dampening immune responses. Through its role in the development of T cell immunologic memory, which depends upon the expansion of the number and function of antigen-selected T cell clones, it plays a key role in enduring cell-mediated immunity.

Aldesleukin is a form of recombinant interleukin-2. It is manufactured using recombinant DNA technology and is marketed as a protein therapeutic and branded as Proleukin. It has been approved by the Food and Drug Administration (FDA) and in several European countries for the treatment of cancers (malignant melanoma, renal cell cancer) in large intermittent doses and has been extensively used in continuous doses.

Interking is a recombinant IL-2 with a serine at residue 125, sold by Shenzhen Neptunus.

Various dosages of IL-2 across the United States and across the world are used. The efficiency and side effects of different dosages is often a point of disagreement. Usually, in the U.S., the higher dosage option is used, affected by cancer type, response to treatment and general patient health. Patients are typically treated for five consecutive days, three times a day, for fifteen minutes. The following approximately 10 days help the patient to recover between treatments. IL-2 is delivered intravenously on an inpatient basis to enable proper monitoring of side effects.

A lower dose regimen involves injection of IL-2 under the skin typically on an outpatient basis. It may alternatively be given on an inpatient basis over 1-3 days, similar to and often including the delivery of chemotherapy. Intralesional IL-2 is commonly used to treat in-transit melanoma metastases and has a high complete response rate and is generally well-tolerated.

IL-2 has a narrow therapeutic window, and the level of dosing usually determines the severity of the side effects. Some common side effects include flu-like symptoms (fever, headache, muscle and joint pain, fatigue), nausea/vomiting, dry, itchy skin or rash, weakness or shortness of breath, diarrhea, low blood pressure, drowsiness or confusion, and loss of appetite. More serious and dangerous side effects sometimes are seen, such as capillary leak syndrome, breathing problems, serious infections, seizures, allergic reactions, heart problems or a variety of other possible complications.

### III. THERAPEUTIC ADMINISTRATION

In another aspect, the present disclosure provides methods of inhibiting the growth or promoting the killing of a tumor cell or treating cancer, such as melanoma, by administering a recombinant oncolytic virus according to the instant disclosure at a multiplicity of infection sufficient to inhibit the growth of a tumor cell or to kill a tumor cell. In certain embodiments, the recombinant oncolytic virus is administered more than once, preferably twice, three times, or up to 10 times.

Examples of tumor cells or cancers that may be treated using the methods of this disclosure include breast cancer, ovarian cancer, renal cell carcinoma (RCC), melanoma (e.g., metastatic malignant melanoma), prostate cancer, colon cancer, lung cancer (including small cell lung cancer and non-small cell lung cancer), bone cancer, osteosarcoma, rhabdomyosarcoma, leiomyosarcoma, chondrosarcoma, pancreatic cancer, skin cancer, fibrosarcoma, chronic or acute leukemias including acute lymphocytic leukemia (ALL), adult T-cell leukemia (T-ALL), acute myeloid leukemia, chronic myeloid leukemia, acute lymphoblastic leukemia, chronic lymphocytic leukemia, lymphangiosarcoma, lymphomas (e.g., Hodgkin's and non-Hodgkin's lymphoma, lymphocytic lymphoma, primary CNS lymphoma, T-cell lymphoma, Burkitt's lymphoma, anaplastic large-cell lymphomas (ALCL), cutaneous T-cell lymphomas, nodular small cleaved-cell lymphomas, peripheral T-cell lymphomas, Lennert's lymphomas, immunoblastic lymphomas, T-cell leukemia/lymphomas (ATLL), entroblastic/centrocytic (cb/cc) follicular lymphomas cancers, diffuse large cell lymphomas of B lineage, angioimmunoblastic lymphadenopathy (AILD)-like T cell lymphoma and HIV associated body cavity based lymphomas), Castleman's disease, Kaposi's Sarcoma, hemangiosarcoma, multiple myeloma, Waldenstrom's macroglobulinemia and other B-cell lymphomas, nasopharangeal carcinomas, head or neck cancer, myxosarcoma, liposarcoma, cutaneous or intraocular malignant melanoma, uterine cancer, rectal cancer, cancer of the anal region, stomach cancer, testicular cancer, uterine cancer, carcinoma of the fallopian tubes, carcinoma of the endometrium, cervical carcinoma, vaginal carcinoma, vulvar carcinoma, transitional cell carcinoma, esophageal cancer, malignant gastrinoma, small intestine cancer, cholangiocellular carcinoma, adenocarcinoma, endocrine system cancer, thyroid gland cancer, parathyroid gland cancer, adrenal gland cancer, sarcoma of soft tissue, urethral, penile cancer, testicular cancer, malignant teratoma, solid tumors of childhood, bladder cancer, kidney or ureter cancer, carcinoma of the renal pelvis, malignant meningioma, neoplasm of the central nervous system (CNS), tumor angiogenesis, spinal axis tumor, pituitary adenoma, epidermoid cancer, squamous cell cancer, environmentally induced cancers including those induced by asbestos, e.g., mesothelioma, and combinations of these cancers. Many cancers overexpress immune checkpoint proteins, such as PDL1 (PDL1⁺). The methods of this disclosure may be used to treat tumors or cancers regardless of PDL1 status.

Oncolytic viruses according to the disclosure may be administered locally or systemically. For example, without limitation, oncolytic viruses according to the disclosure can be administered intravascularly (intraarterially or intravenously), intratumorally, intramuscularly, intradermally, intraperitoneally, subcutaneously, orally, parenterally, intranasally, intratracheally, percutaneously, intraspinally, ocularly, or intracranially.

In still another embodiment, the methods involve parenteral administration of a recombinant oncolytic virus, preferably via an artery or via an in-dwelling medical device. The recombinant oncolytic virus can be administered with an immunotherapeutic agent or immunomodulator, such as an antibody that binds to a tumor-specific antigen (e.g., chimeric, humanized or human monoclonal antibodies). In another embodiment, the recombinant oncolytic virus treatment may be combined with surgery (e.g., tumor excision), radiation therapy, chemotherapy, or immunotherapy, and can be administered before, during or after a complementary treatment.

In other embodiments, the method involves ex vivo transduction of cells with a myxoma virus of the present invention, followed by administration of a composition comprising the cells into a subject. In certain embodiments, the cells may be autologous, i.e., the subject's own cells. In autologous embodiments, the cells may be obtained from the subject, transduced with a myxoma virus of the present invention, and re-administered into the subject, in a process similar to apheresis. Exemplary formulations for ex vivo delivery of the virus into cells may include the use of various transduction agents known in the art, such as calcium phosphate, electoporation, heat shock and various liposome formulations (i.e., lipid-mediated transfection). Liposomes, as described in greater detail below, are lipid bilayers entrapping a fraction of aqueous fluid. DNA spontaneously associates to the external surface of cationic liposomes (by virtue of its charge) and these liposomes will interact with the cell membrane.

In certain embodiments, the recombinant oncolytic virus and an immunotherapeutic agent or immunomodulator can be administered concurrently or sequentially in a way that the agent does not interfere with the activity of the virus. In certain embodiments, the recombinant oncolytic virus is administered intra-arterially, intravenously, intraperitoneally, intratumorally, or any combination thereof. In still another embodiment, an interferon, such as interferon-a or pegylated interferon, is administered prior to administering the recombinant oncolytic virus according to the instant disclosure.

Oncolytic viruses according to the disclosure may be administered in a single administration or multiple administrations. The virus may be administered at dosage of 1×10⁵ plaque forming units (PFU), 5×10⁵ PFU, at least 1×10⁶ PFU, 5×10⁶ or about 5×10⁶ PFU, 1×10⁷, at least 1×10⁷ PFU, 1×10⁸ or about 1×10⁸ PFU, at least 1×10⁸ PFU, about or at least 5×10⁸ PFU, 1×10⁹ or at least 1×10⁹ PFU, 5×10⁹ or at least 5×10⁹ PFU, 1×10¹⁰ PFU or at least 1×10¹⁰ PFU, 5×10¹⁰ or at least 5×10¹⁰ PFU, 1×10¹¹ or at least 1×10¹¹, 1×10¹² or at least 1×10¹², 1×1 0¹³ or at least 1×10¹³. For example, the virus may be administered at a dosage of between about 10⁷-1 0¹³, between about 10⁸-10¹³, between about 10⁹-10¹², or between about 10⁸-10¹².

### A. Combination Therapies

Additional therapies may be combined with any of the methods of the disclosure heretofore described in order to increase the killing of cancer cells, the inhibition of cancer cell growth, the inhibition of angiogenesis or otherwise improve the reverse or reduction of malignant phenotype of tumor cells. These compositions would be provided in a combined amount effective to kill or inhibit proliferation of the cell. This process may involve contacting the cells with the expression construct and the agent(s) or factor(s) at the same time. This may be achieved by contacting the cell with a single composition or pharmacological formulation that includes both agents, or by contacting the cell with two distinct compositions or formulations, at the same time, wherein one composition includes the oncolytic virus and the other includes a second agent therapy.

Alternatively, the treatment may precede or follow the other agent or treatment by intervals ranging from minutes to weeks. In embodiments where the agents are applied separately to the cell, one would generally ensure that a significant period of time did not expire between each delivery, such that the agents would still be able to exert an advantageously combined effect on the cell. In such instances, it is contemplated that one would contact the cell with both modalities within about 12-24 hours of each other and, more preferably, within about 6-12 hours of each other, with a delay time of only about 12 hours being most preferred. In some situations, it may be desirable to extend the time period for treatment significantly, however, where several days (2, 3, 4, 5, 6 or 7) to several weeks (1, 2, 3, 4, 5, 6, 7 or 8) to several months (1, 2, 3, 4, 5, 6, 7 or 8) lapse between the respective administrations.

It also is conceivable that more than one administration of either agent will be desired. Various combinations may be employed, e.g. where one or more oncolytic virus treatment is administered before the administration of a second agent; or the second agent may be administered prior to oncolytic virus administration. Successive administration can include one or more administration of the oncolytic virus therapy or second agent. Again, to achieve cell killing, both agents are delivered to a cell in a combined amount effective to kill the cell. For example, the combination of the claimed PD1+IL-2/IL-12 reagent and an immune modulator.

In accordance with certain embodiments of the present disclosure, methods for treating cancer are provided that can be used in conjunction with oncolytic virus therapy once a subject is identified as a responder or likely to respond to such therapy (e.g. vMYX-PD1 therapy). Such therapies may be utilized when the assays of the present disclosure indicate that a subject is unlikely to respond to treatment with a replication competent oncolytic virus such as myxoma virus. Alternatively, such therapies may be utilized in combination with replication competent oncolytic virus such as adenovirus in the case that a subject is identified by the present methods as unlikely to respond to treatment with only replication competent oncolytic virus.

Approximately 60% of persons with cancer will undergo surgery of some type, which includes preventative, diagnostic, staging, curative and palliative surgery. Curative surgery is a cancer treatment that may be used in conjunction with other therapies, such as the treatment of the present disclosure, chemotherapy, radiotherapy, hormonal therapy, gene therapy, immunotherapy and/or alternative therapies.

Curative surgery includes resection in which all or part of cancerous tissue is physically removed, excised and/or destroyed. Tumor resection refers to physical removal of at least part of a tumor. In addition to tumor resection, treatment by surgery includes laser surgery, cryosurgery, electrosurgery, and microscopically controlled surgery (Mohs' surgery). It is further contemplated that the present disclosure may be used in conjunction with removal of superficial cancers, precancers, or incidental amounts of normal tissue.

In certain aspects, a therapy is administered by intratumoral injection prior to surgery or upon excision of a part of or all of cancerous cells, tissue or tumor. Treatment may also be accomplished by perfusion, direct injection or local application of these areas with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages.

A wide variety of chemotherapeutic agents may be used in accordance with the present disclosure. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis. Most chemotherapeutic agents fall within the following categories: alkylating agents, antimetabolites, antitumor antibiotics, topoisomerase inhibitors, and mitotic inhibitors.

Alkylating agents direct interact with genomic DNA to prevent the cancer cell from proliferating. This category of drugs includes agents that affect all phases of the cell cycle and are commonly used to treat chronic leukemia, non-Hodgkin's lymphoma, Hodgkin's disease, malignant melanoma, multiple myeloma, and particular cancers of the breast, lung, and ovary. They include nitrogen mustards such as mechlorethamine (nitrogen mustard), chlorambucil, cyclophosphamide (Cytoxan®), ifosfamide and melphalan, nitrosoureas such as streptozocin, carmustine (BCNU) and lomustine, alkyl sulfonates such as busulfan, triazines such as dacarbzine (DTIC) and temozolomide (Temodar®), ethylenimines such as thiotepa and altretamine (hexamethylmelamine), and platinum drugs such as cisplatin, carboplatin, and oxalaplatin.

Antimetabolites disrupt DNA and RNA synthesis. Unlike alkylating agents, they specifically influence the cell cycle during S phase. They have been used to combat chronic leukemias, and tumors of the breast, ovary and gastrointestinal tract. Antimetabolites include 5-fluorouracil (5-FU), 6-mercaptopurine (6-MP), capecitabine (Xelode), cladribine, clofarabine, cytarabine (Ara-C®), floxuridine, fludarabine, gemcitabine (Gemzar®), hydroxyruea, methotrexate, pemetrexed, pentostatin and thioguanine.

Antitumor antibiotics have both antimicrobial and cytotoxic activity. These drugs also interfere with DNA by chemically inhibiting enzymes and mitosis or altering cellular membranes. These agents work in all phases of the cell cycle and are used to treat a variety of cancers. Representative examples include daunorubicin, doxorubicin (Adriamycin®), epirubicin, idarubicin, actinomycin-D, bleomycin and mitomycin-C. Generally, these compounds are administered by bolus i.v. injections at doses ranging from 25-100 mg/kg

Topoisomerase inhibitors interfere with topoisomerases, enzymes which help separate DNA strands so they can be copied and are used to treat certain leukemias, as well as lung, ovarian, gastrointestinal and other cancers and include topotecan, irinotecan, etoposide (VP-16) and teniposide.

Mitotic inhibitors, often plant alkaloids, work during M phase of the cell cycle and prevent mitosis or inhibit enzymes from producing proteins required for cell reproduction. Representative examples include taxanes such as paclitaxel (Taxol®) and docetaxel (Taxotere®), epothilones such as ixabepilone (Ixempra®), vinca alkaloids such as vinblastine (Velban®), vincristine (Onocovin®) and vinorelbine (Navelbin®), and Estramustine (Emcyt®).

In some embodiments, immunotherapy may be treatment with an immune checkpoint inhibitor. Immune checkpoints either turn up a signal (e.g., co-stimulatory molecules) or turn down a signal. Inhibitory immune checkpoints that may be targeted by immune checkpoint blockade include adenosine A2A receptor (A2AR), B7-H3 (also known as CD276), B and T lymphocyte attenuator (BTLA), cytotoxic T-lymphocyte-associated protein 4 (CTLA-4, also known as CD152), indoleamine 2,3-dioxygenase (IDO), killer-cell immunoglobulin (KIR), lymphocyte activation gene-3 (LAG3), programmed death 1 (PD-1), T-cell immunoglobulin domain and mucin domain 3 (TIM-3) and V-domain Ig suppressor of T cell activation (VISTA).

The immune checkpoint inhibitors may be drugs such as small molecules, recombinant forms of ligand or receptors, or, in particular, are antibodies, such as human antibodies directed to the immune checkpoint proteins (e.g., International Patent Publication WO2015016718; Pardoll, 2012; both incorporated herein by reference). Known inhibitors of the immune checkpoint proteins or analogs thereof may be used, in particular chimerized, humanized or human forms of antibodies may be used. As the skilled person will know, alternative and/or equivalent names may be in use for certain antibodies mentioned in the present disclosure. Such alternative and/or equivalent names are interchangeable in the context of the present disclosure. For example it is known that lambrolizumab is also known under the alternative and equivalent names MK-3475 and pembrolizumab. Exemplary immune checkpoint inhibitors include PD-1 inhibitors, such as Pembrolizumab and Nivolumab; PD-L1 inhibitors, such as Atezolizumab, Avelumab, and Durvalumab; and CTLA-4 inhibitors, such as Ipilimumab.

In certain preferred embodiments, additive anti-tumor effects can be achieved by combining myxoma vPD1 with blockade of PD1 on T-cells directly. Clinically, this may be achieved through the use of antibodies that bind PD1 blocking interaction with PDL1. It is observed clinically that combination of individual immune checkpoint inhibition achieves much better antitumor activity (Johnson and Win, 2017, incorporated herein by reference in its entirety). An additional benefit of combing myxoma vPD1 and anti-PD1 antibodies according to the present invention may be in the setting of metastatic disease. Locally administered myxoma vPD1, via intra-tumoral injection, may not be optimal for metastatic disease due to PK/PD issues.

While combining the oncolytic viruses according to the present invention with anti-PD1 antibodies is a promising approach, possible complications might occur through interaction of the anti-PD1 antibody and soluble PD1 expressed from the myxoma virus. To ameliorate this possibility, a myxoma virus was produced expressing a PD1 construct containing mutations in the CD loop that prevents antibody recognition between the two clinically approved anti-PD1 antibodies. In one embodiment, a site mutation at position D85G in the PD1 protein will completely abolish the binding of anti-PD1 antibody pembrolizumab to PD1 (Tan et al., 2017, incorporated herein by reference in its entirety; and Na et al., incorporated herein by reference in its entirety). Thus, in this embodiment, introducing a single point mutation or combinations of single point mutations between the CD loop in the truncated PD1 myxoma construct will decrease any inhibitory binding of anti-PD1 antibody.

Other chemotherapeutic agents include targeted therapies such as imatinib (Gleevec®), gefitinib (Iressa®), sunitinib (Sutent®), sorafenib (Nexavar®), bortezomib (Velcade®), bevacizumab (Avastin®), trastuzumab (Herceptin®), cetuximab (Erbitux®), and panitumumab (Vectibix®), hormone therapies including antiestrogens such as fulvestrant (Faslodex®), tamoxifen, toremifene, aromatase inhibitors such as anastrozole, exemstane and letrozole, progestins such as megestrol acetate, and gonadotropin-releasing hormone and immunotherapies such as antibodies against tumor specific antigens (e.g. prostate specific antigen, carcinoembryonic antigen, urinary tumor associated antigen, fetal antigen, tyrosinase (p97), gp68, TAG-72, HMFG, Sialyl Lewis Antigen, MucA, MucB, PLAP, estrogen receptor, laminin reeptor, erb B and p155) which may be conjugated to a drug or toxin (e.g. radionuclide, ricin A chain, cholera toxin, pertussis toxin).

Radiotherapy, also called radiation therapy, is the treatment of cancer and other diseases with ionizing radiation which may be used to treat localized solid tumors such as cancers of the skin, tongue, larynx, brain, breast or cervix, or may be used to treat cancers of the blood-forming cells (leukemia) and lymphatic system (lymphoma). Radiation therapy includes, without limitation, the use of y-rays, X-rays and/or the directed delivery of radioisotopes to tumor cells. Other forms of DNA damaging factors are contemplated such as microwaves and UV-irradiation. Dosage ranges for X-rays range from daily doses of 50-200 roentgens for prolonged periods of time (3 to 4 weeks), to single doses of 2000-6000 roentgens.

Radiotherapy also comprises the use of radiolabeled antibodies to deliver doses of radiation directly to the cancer site (e.g. radioimmunotherapy, conformal radiotherapy), high resolution intensity modulated radiotherapy, and stereotactic radio-surgery. Stereotactic radio-surgery (gamma knife) for brain and other tumors employs precisely targeted beams of gamma radiotherapy from hundreds of different angles. Only one session, taking about 4-5 hours is required.

### B. Pharmaceutical Compositions

The recombinant oncolytic virus described herein can be administered as a pharmaceutical or medicament formulated with a pharmaceutically acceptable carrier. Accordingly, the recombinant oncolytic virus may be used in the manufacture of a medicament or pharmaceutical composition. Pharmaceutical compositions of the disclosure may be formulated as solutions or lyophilized powders for parenteral administration. Powders may be reconstituted by addition of a suitable diluent or other pharmaceutically acceptable carrier prior to use. Liquid formulations may be buffered, isotonic, aqueous solutions. Powders also may be sprayed in dry form. Examples of suitable diluents are normal isotonic saline solution, standard 5% dextrose in water, or buffered sodium or ammonium acetate solution. Such formulations are especially suitable for parenteral administration, but may also be used for oral administration or contained in a metered dose inhaler or nebulizer for insufflation. It may be desirable to add excipients such as polyvinylpyrrolidone, gelatin, hydroxy cellulose, acacia, polyethylene glycol, mannitol, sodium chloride, sodium citrate, and the like.

Alternately, therapeutic agents may be encapsulated, tableted or prepared in an emulsion or syrup for oral administration. Pharmaceutically acceptable solid or liquid carriers may be added to enhance or stabilize the composition, or to facilitate preparation of the composition. Solid carriers include starch, lactose, calcium sulfate dihydrate, terra alba, magnesium stearate or stearic acid, talc, pectin, acacia, agar or gelatin. Liquid carriers include syrup, peanut oil, olive oil, saline and water. The carrier may also include a sustained release material such as glyceryl monostearate or glyceryl distearate, alone or with a wax. The amount of solid carrier varies but, preferably, will be between about 20 mg to about 1 g per dosage unit. The pharmaceutical preparations are made following the conventional techniques of pharmacy involving milling, mixing, granulating, and compressing, when necessary, for tablet forms; or milling, mixing and filling for hard gelatin capsule forms. When a liquid carrier is used, the preparation may be in the form of a syrup, elixir, emulsion, or an aqueous or non-aqueous suspension. For rectal administration, the disclosure compounds may be combined with excipients such as cocoa butter, glycerin, gelatin, or polyethylene glycols and molded into a suppository.

Therapeutic agents may be formulated to include other medically useful drugs or biological agents. The therapeutic agents also may be administered in conjunction with the administration of other drugs or biological agents useful for the disease or condition to which the disclosure compounds are directed.

The biologic or pharmaceutical compositions of the present disclosure can be formulated to allow the recombinant oncolytic virus contained therein to be bioavailable upon administration of the composition to a subject. The level of recombinant oncolytic virus in serum, tumors, and other tissues after administration can be monitored by various well-established techniques, such as antibody-based assays (e.g., ELISA). In certain embodiments, recombinant oncolytic virus compositions are formulated for parenteral administration to a subject in need thereof (e.g., a subject having a tumor), such as a non-human animal or a human. Preferred routes of administration include intravenous, intra-arterial, subcutaneous, intratumoral, or intramuscular.

Proper formulation is dependent upon the route of administration chosen, as is known in the art. For example, systemic formulations are an embodiment that includes those designed for administration by injection, e.g., subcutaneous, intra-arterial, intravenous, intramuscular, intrathecal or intraperitoneal injection, as well as those designed for intratumoral, transdermal, transmucosal, oral, intranasal, or pulmonary administration. In one embodiment, the systemic or intratumoral formulation is sterile. In embodiments for injection, the recombinant oncolytic virus compositions of the instant disclosure may be formulated in aqueous solutions, or in physiologically compatible solutions or buffers such as Hanks's solution, Ringer's solution, mannitol solutions or physiological saline buffer. In certain embodiments, any of the recombinant oncolytic virus compositions described herein may contain formulator agents, such as suspending, stabilizing or dispersing agents. In embodiments for transmucosal administration, penetrants, solubilizers or emollients appropriate to the harrier to be permeated may be used in the formulation. For example, 1-dodecylhexahydro-2H-azepin-2-one (Azon®), oleic acid, propylene glycol, menthol, diethyleneglycolethoxyglycol monoethyl ether (Transcutol®), polysorbate polyethylenesorbitan monolaurate (Tween®-20), and the drug 7-chloro-1-methyl-5-phenyl-3H-1,4-benzodiazepin-2-one (Diazepam), isopropyl myristate, and other such penetrants, solubilizers or emollients generally known in the art may be used in any of the compositions of the instant disclosure.

Administration can be achieved using a combination of routes, e.g., first administration using an intra-arterial route and subsequent administration via an intravenous or intratumoral route, or any combination thereof.

### IV. EXAMPLES

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1—Generation and Characterization of vMYX-PD1 Constructs

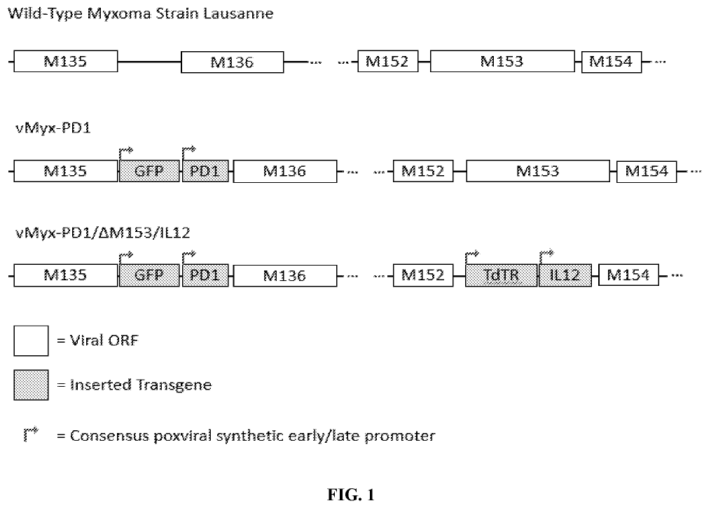

Recombinant virus construct may be made with soluble PD1 or soluble PD1 and optionally various interleukins. See, e.g., the schematics of a representative recombinant viral genomic structure in FIG. 1.

To construct the vPD1 and mutant vPD1, the extracellular region of human PD1 (amino acids 1-168) was amplified from a preconstructed template plasmid (PlasmID database, clone HsCD00345685) by PCR using the following primers.

Forward Primer:
[SEQ ID NO: 1]
ATCGCCCGGGAAAAATTGAAATTTTATTTTTTTTTTTTGGAATATAAATA
ACCATGCAGATCCCACAGGCGCC
Reverse Primer:
[SEQ ID NO: 2]
ATCGGAATTCTCAGGTTTGGAACTGGCCGGCTG
Soluble PD1 nucleotide sequence:
[SEQ ID NO: 3]
ATGCAGATCCCACAGGCGCCCTGGCCAGTCGTCTGGGCGGTGCTACAACT
GGGCTGGCGGCCAGGATGGTTCTTAGACTCCCCAGACAGGCCCTGGAACC
CCCCCACCTTCTCCCCAGCCCTGCTCGTGGTGACCGAAGGGGACAACGCC
ACCTTCACCTGCAGCTTCTCCAACACATCGGAGAGCTTCGTGCTAAACTG
GTACCGCATGAGCCCCAGCAACCAGACGGACAAGCTGGCCGCTTTCCCCG
AGGACCGCAGCCAGCCCGGCCAGGACTGCCGCTTCCGTGTCACACAACTG
CCCAACGGGCGTGACTTCCACATGAGCGTGGTCAGGGCCCGGCGCAATGA
CAGCGGCACCTACCTCTGTGGGGCCATCTCCCTGGCCCCCAAGGCGCAGA
TCAAAGAGAGCCTGCGGGCAGAGCTCAGGGTGACAGAGAGAAGGGCAGAA
GTGCCCACAGCCCACCCCAGCCCCTCACCCAGGCCAGCCGGCCAGTTCCA
AACC
Native Soluble PD1 amino acid sequence (1-168)
Q15116 20 amino signal peptide
[SEQ ID NO: 4]
MQIPQAPWPV VWAVLQLGWR PGWFLDSPDR PWNPPTFSPA
LLVVTEGDNA TFTCSFSNTS ESFVLNWYRM SPSNQTDKLA
AFPEDRSQPG QDCRFRVTQL PNGRDFHMSV VRARRNDSGT
YLCGAISLAP KAQIKESLRA ELRVTERRAE VPTAHPSPSP
RPAGQFQT
Mutated Soluble PD1 amino acid sequence (1-168)
(D85G substitution abolishes the binding of
pembrolizumab to PD1)
20 amino signal peptide
[SEQ ID NO: 5]
MQIPQAPWPV VWAVLQLGWR PGWFLDSPDR PWNPPTFSPA
LLVVTEGDNA TFTCSFSNTS ESFVLNWYRM SPSNQTDKLA
AFPEGRSQPG QDCRFRVTQL PNGRDFHMSV VRARRNDSGT
YLCGAISLAP KAQIKESLRA ELRVTERRAE VPTAHPSPSP
RPAGQFQT
Human IL-2 amino acid sequence (1-153)
P60568 20 amino signal peptide
[SEQ ID NO: 6]
MYRMQLLSCI ALSLALVTNS APTSSSTKKT QLQLEHLLLD
LQMILNGINN YKNPKLTRML TFKFYMPKKA TELKHLQCLE
EELKPLEEVL NLAQSKNFHL RPRDLISNIN VIVLELKGSE
TTFMCEYADE TATIVEFLNR WITFCQSIIS TLT
Human IL-12, subunit alpha amino acid sequence
(1-219)
P29459 22 amino signal peptide
[SEQ ID NO: 7]
MCPARSLLLV ATLVLLDHLS LARNLPVATP DPGMFPCLHH
SQNLLRAVSN MLQKARQTLE FYPCTSEEID HEDITKDKTS
TVEACLPLEL TKNESCLNSR ETSFITNGSC LASRKTSFMM
ALCLSSIYED LKMYQVEFKT MNAKLLMDPK RQIFLDQNML
AVIDELMQAL NFNSETVPQK SSLEEPDFYK TKIKLCILLH
AFRIRAVTID RVMSYLNAS
Human IL-12, subunit beta amino acid sequence
(1-328)
P29460 22 amino signal peptide
[SEQ ID NO: 8]
MCHQQLVISW FSLNFLASPL VA IWELKKDV YVVELDWYPD
APGEMVVLTC DTPEEDGITW TLDQSSEVLG SGKTLTIQVK
EFGDAGQYTC HKGGEVLSHS LLLLHKKEDG IWSTDILKDQ
KEPKNKTFLR CEAKNYSGRF TCWWLTTIST DLTFSVKSSR
GSSDPQGVTC GAATLSAERV RGDNKEYEYS VECQEDSACP
AAEESLPIEV MVDAVHKLKY ENYTSSFFIR DIIKPDPPKN
LQLKPLKNSR QVEVSWEYPD TWSTPHSYFS LTFCVQVQGK
SKREKKDRVF TDKTSATVIC RKNASISVRA QDRYYSSSWS
EWASVPCS
High Affinity Human IL-2 amino acid variant
(1-164)
P60568 20 amino signal peptide
[SEQ ID NO: 9]
MYRMQLLSCI ALSLALVTNS APTSSSTKKT QLQLEHLLLD
LQMILNGINN YKNPKLTRML TFKFYMPKKA TELKHLQCLE
EELKPLEEVL NLAQSKNFHF DPRDVVSNIN VFVLELKGSE
TTFMCEYADE TATIVEFLNR WITFCQSIIS TLTAAAHHHH
HHHH

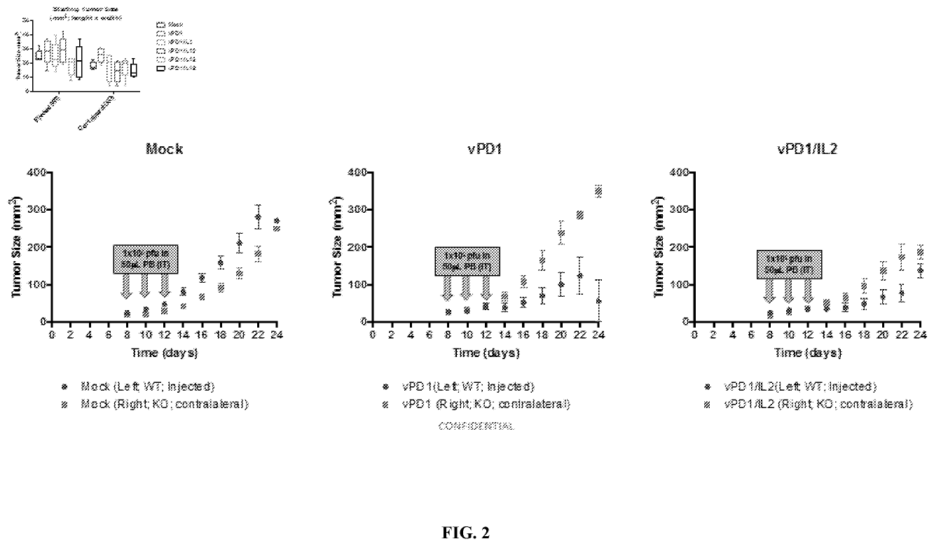

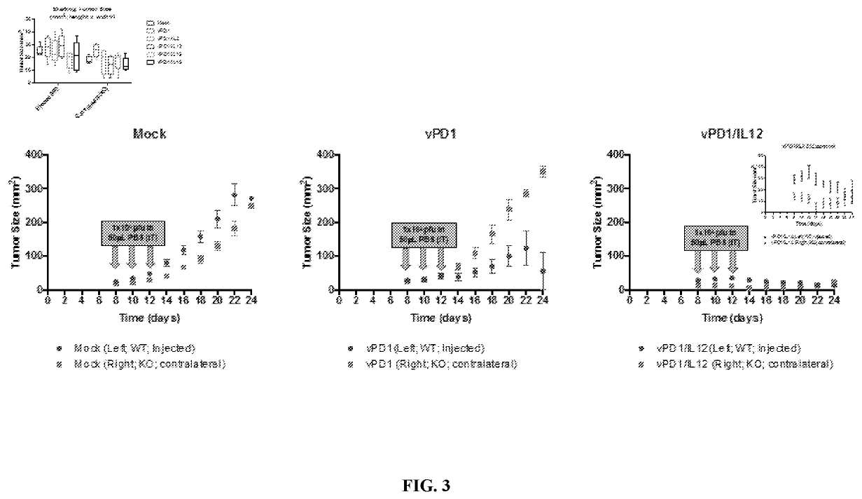

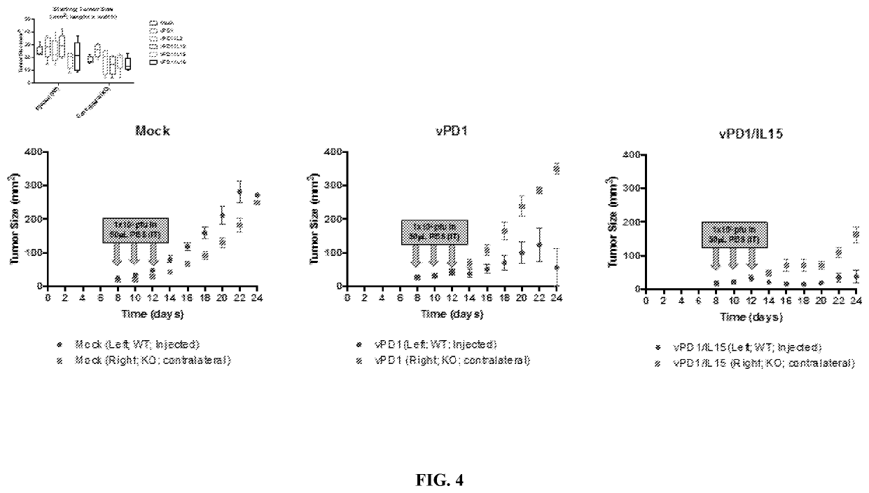

FIG. 2 shows a vPD1-IL2 efficacy study in subcutaneous B16F10 (B16F10 PD1L-KO) contralateral xenograft model. FIG. 3 shows a vPD1-IL12 efficacy study in subcutaneous B16F10 (B16F10 PD1L-KO) contralateral xenograft model. FIG. 4 shows the results of a vPD1-IL15 efficacy study in subcutaneous B16F10 (B16F10 PD1L-KO) contralateral xenograft model. Likewise, FIG. 5 shows results from a vPD1-IL18 efficacy study in subcutaneous B16F10 (B16F10 PD1L-KO) contralateral xenograft model. Taken together, IL-2 and IL-15 appear to show modest reductions in tumor size, while IL-12 provides the greatest reduction. IL-18 appears to have the least effect.

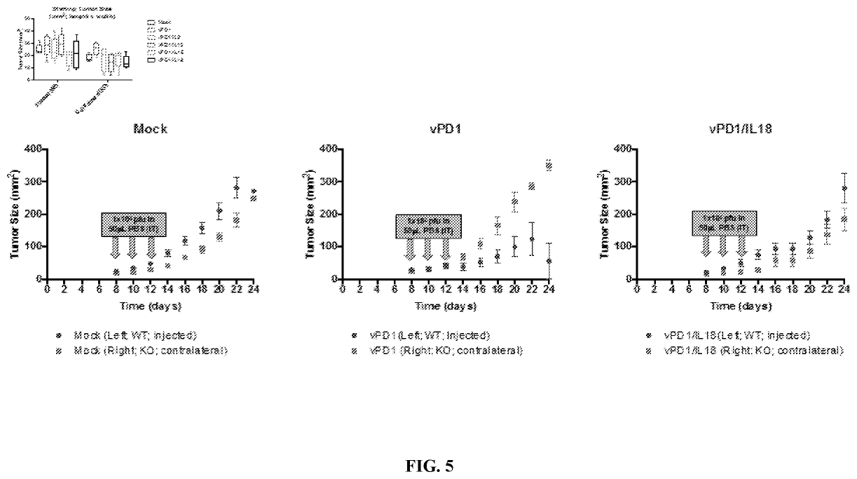

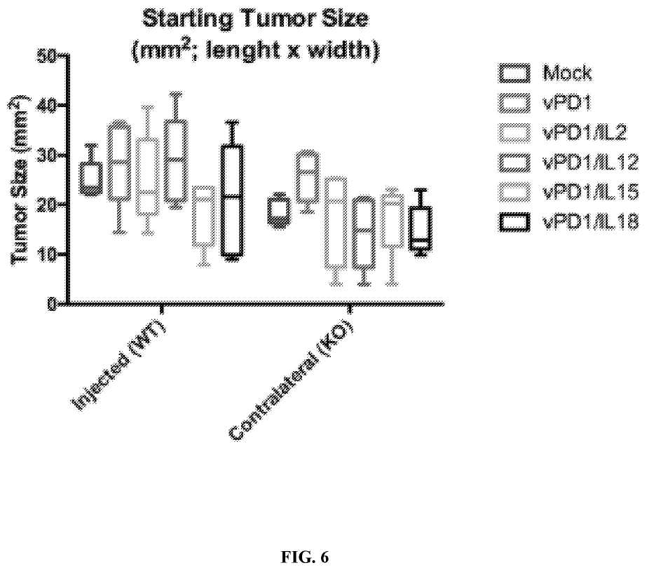

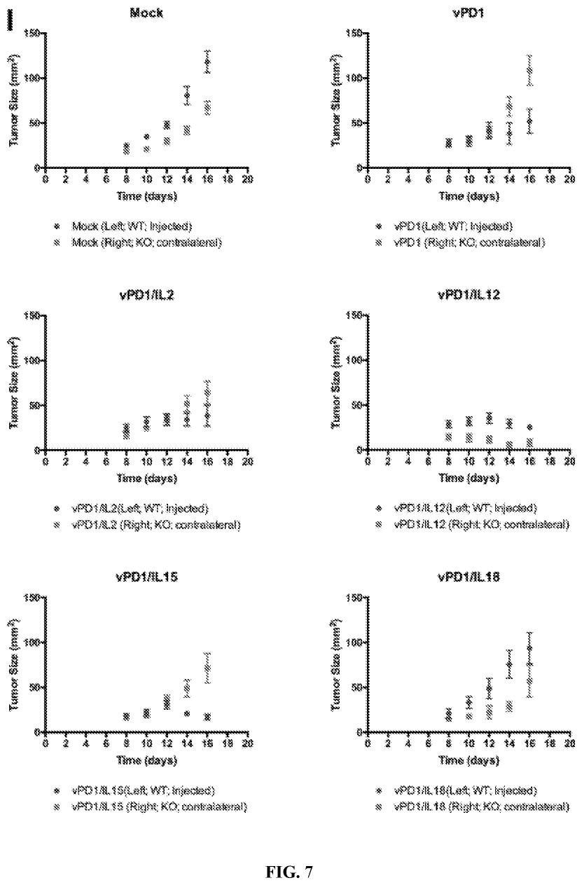

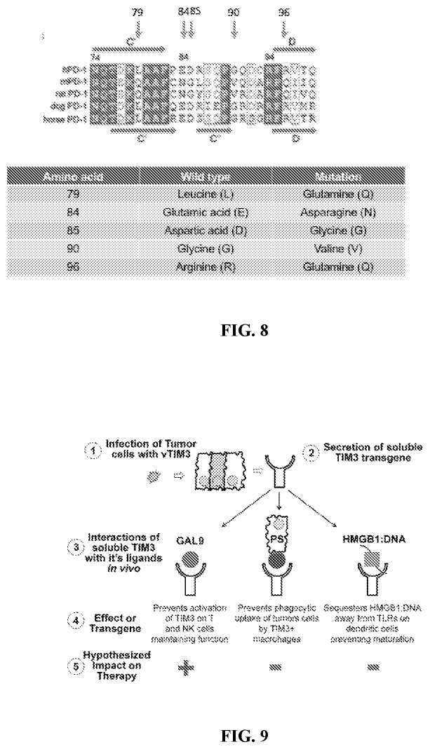

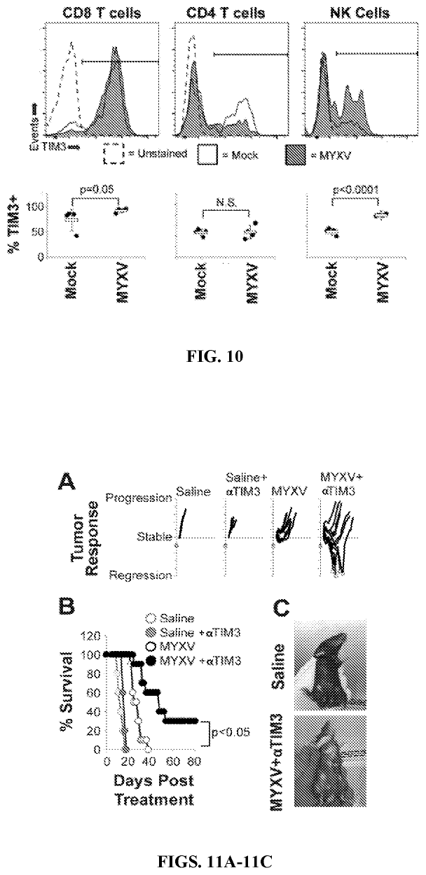

As shown in FIGS. 6 and 7, an in vivo subcutaneous (SC) contralateral mouse model was employed to test various viral constructs. Three intratumoral injections were made two days apart on the left side (WT-B16/F10) while the right side (PDL1-KO-B16/F10) was untreated. Injected tumors (left) and contralateral non-injected tumors (right) seem to respond to vPD1/IL12 and vPD1/IL2 treatments. vPD1/IL15 showed a modest esponse as well, whereas vPD/IL18 show negligible effect. Taken together, IL-12 surprisingly shows a greater reduction in the size of both the injected and the contralateral tumors than do the other constructs tested.

TABLE 1
RAW VIRUS TREATMENT DATA

| Treatment | | Animal | Admin | Day 8 | Day 10 | Day 12 | Day 14 | Day 16 |
|---|---|---|---|---|---|---|---|---|
| Mock | 1L | WT | Injected | 23.52 | 35.96 | 57.6 | 91.18 | 112.11 |
| Mock | 2L | WT | Injected | 22.96 | 32.83 | 43.56 | 113.46 | 151.51 |
| Mock | 3L | WT | Injected | 24.44 | 37.12 | 35.99 | 55.2 | 77.7 |
| Mock | 4L | WT | Injected | 22.05 | 26.95 | 49.64 | 69.3 | 123.42 |
| Mock | 5L | WT | Injected | 31.92 | 40.8 | 52.65 | 74.25 | 126.26 |
| Mock | 1R | KO | Contralateral | 17.22 | 16.92 | 29.15 | 45.14 | 85.36 |
| Mock | 2R | KO | Contralateral | 15.58 | 17.63 | 26.5 | 32.76 | 56.21 |
| Mock | 3R | KO | Contralateral | 20.16 | 19.68 | 21.12 | 31.92 | 57 |
| Mock | 4R | KO | Contralateral | 17.16 | 23 | 30.09 | 42.78 | 53.13 |
| Mock | 5R | KO | Contralateral | 22.05 | 28.05 | 42.48 | 56.7 | 83.66 |
| vPD1 | 1L | WT | Injected | 34.79 | 38.5 | 60.75 | 78.54 | 92.13 |
| vPD1 | 2L | WT | Injected | 14.43 | 11.22 | 11.22 | 6 | 11.55 |
| vPD1 | 3L | WT | Injected | 27.93 | 37.26 | 45.99 | 46.08 | 62.64 |
| vPD1 | 4L | WT | Injected | 28.62 | 34.22 | 58.855 | 33.6 | 55.68 |
| vPD1 | 5L | WT | Injected | 36.6 | 25.97 | 32.76 | 27.44 | 38.35 |
| vPD1 | 1R | KO | Contralateral | 22.95 | 23.5 | 36.58 | 63.75 | 105.06 |
| vPD1 | 2R | KO | Contralateral | 18.48 | 20.16 | 26.52 | 40.32 | 61.6 |
| vPD1 | 3R | KO | Contralateral | 26.52 | 35.4 | 31.8 | 51.84 | 86.49 |
| vPD1 | 4R | KO | Contralateral | 30.6 | 42.25 | 55.44 | 96.03 | 147.84 |
| vPD1 | 5R | KO | Contralateral | 29.7 | 36 | 51.12 | 90.25 | 141.52 |
| vPD1/IL2 | 1L | WT | Injected | 22 | 32.86 | 44.53 | 55.25 | 74.48 |
| vPD1/IL2 | 2L | WT | Injected | 26.5 | 38.86 | 37.8 | 21.5 | 17.22 |
| vPD1/IL2 | 3L | WT | Injected | 14.28 | 17.2 | 10.56 | 15.84 | 11.2 |
| vPD1/IL2 | 4L | WT | Injected | 22.5 | 23.52 | 34.1 | 39.2 | 49.14 |
| vPD1/IL2 | 5L | WT | Injected | 39.68 | 46.8 | 43.55 | 39.04 | 40.3 |
| vPD1/IL2 | 1R | KO | Contralateral | 25.3 | 29.5 | 48.84 | 66.36 | 88.35 |
| vPD1/IL2 | 2R | KO | Contralateral | 24.99 | 25.38 | 32.94 | 40.8 | 50.05 |
| vPD1/IL2 | 3R | KO | Contralateral | 10.88 | 30.09 | 41.58 | 68.06 | 94.05 |
| vPD1/IL2 | 4R | KO | Contralateral | 4 | 16.4 | 20.7 | 23.52 | 26.01 |
| vPD1/IL2 | 5R | KO | Contralateral | 20.7 | 20.68 | 36 | 61.2 | 63.18 |
| vPD1/IL12 | 1L | WT | Injected | 19.5 | 20.24 | 34.2 | 15.54 | 28.52 |
| vPD1/IL12 | 2L | WT | Injected | 29 | 29.76 | 23.97 | 36.54 | 22.88 |
| vPD1/IL12 | 3L | WT | Injected | 22.09 | 24.99 | 24.01 | 19.35 | 15.64 |
| vPD1/IL12 | 4L | WT | Injected | 31.27 | 44.1 | 54.72 | 39.53 | 30 |
| vPD1/IL12 | 5L | WT | Injected | 42.25 | 39.76 | 39.76 | 36.5 | 28.91 |
| vPD1/IL12 | 1R | KO | Contralateral | 4 | 0 | 14.26 | 0 | 0 |
| vPD1/IL12 | 2R | KO | Contralateral | 14.8 | 12.48 | 0 | 0 | 22.05 |
| vPD1/IL12 | 3R | KO | Contralateral | 20.4 | 22.96 | 14.35 | 0 | 0 |
| vPD1/IL12 | 4R | KO | Contralateral | 21.42 | 22.95 | 22.09 | 18.62 | 11.84 |
| vPD1/IL12 | 5R | KO | Contralateral | 10.85 | 7.56 | 7.54 | 7 | 6 |
| vPD1/IL15 | 1L | WT | Injected | 7.84 | 13.02 | 24.96 | 11.88 | 8.99 |
| vPD1/IL15 | 2L | WT | Injected | 21.12 | 21.56 | 33.06 | 19.8 | 14.04 |
| vPD1/IL15 | 3L | WT | Injected | 15.96 | 10.15 | 19.78 | 18.48 | 14 |
| vPD1/IL15 | 4L | WT | Injected | 23.46 | 25.5 | 46.8 | 28.6 | 23.22 |
| vPD1/IL15 | 5L | WT | Injected | 23.46 | 35.4 | 29.12 | 25.44 | 24.48 |
| vPD1/IL15 | 1R | KO | Contralateral | 4 | 9.8 | 18.33 | 23.5 | 40.26 |
| vPD1/IL15 | 2R | KO | Contralateral | 20.7 | 24.5 | 38.5 | 48.51 | 64.6 |
| vPD1/IL15 | 3R | KO | Contralateral | 20.24 | 19.27 | 39.65 | 48.3 | 63.99 |
| vPD1/IL15 | 4R | KO | Contralateral | 23 | 30.24 | 48.18 | 80.84 | 134.82 |
| vPD1/IL15 | 5R | KO | Contralateral | 19.27 | 27.5 | 38.4 | 43.55 | 53.25 |
| vPD1/IL18 | 1L | WT | Injected | 21.62 | 36.58 | 55.89 | 116 | |
| vPD1/IL18 | 2L | WT | Injected | 36.6 | 52.56 | 83.7 | 101.01 | 122.72 |
| vPD1/IL18 | 3L | WT | Injected | 26.95 | 39.04 | 56.07 | 80.64 | 123.12 |
| vPD1/IL18 | 4L | WT | Injected | 10.88 | 20.68 | 24.5 | 39.65 | 57.72 |
| vPD1/IL18 | 5L | WT | Injected | 9 | 16.81 | 24.08 | 40.87 | 71.2 |
| vPD1/IL18 | 1R | KO | Contralateral | 15.6 | 15.99 | 20.09 | 27.56 | |
| vPD1/IL18 | 2R | KO | Contralateral | 12.95 | 17.766 | 17.55 | 22.36 | 51.62 |
| vPD1/IL18 | 3R | KO | Contralateral | 12.24 | 12.58 | 10.56 | 18.92 | 33.63 |
| vPD1/IL18 | 4R | KO | Contralateral | 9.9 | 14.4 | 12.8 | 24.36 | 34.8 |
| vPD1/IL18 | 5R | KO | Contralateral | 22.95 | 27.54 | 50.37 | 49.56 | 108.81 |

As shown in FIG. 15, a mouse study was performed to assess the efficacy of vPD1 alone, vIL12 alone, and the combination of vPD1+IL12. Mice were injected with 4×10⁶ B16/F10 cells on both flanks. After tumors were established, the larger tumor was treated with 3 injections of the indicated virus over 5 days (Day 0, 2, and 4). The growth of the tumors and the body weight of the mice were monitored until the mice were euthanized when the total tumor burden exceeded 400 mm². It was found that the combination of vPD1+IL12 had the most significant effect on decreasing tumor growth and increasing overall survival of the mice.

Further development of the virus comprised the addition of a transmembrane domain to the IL12. The transmembrane domain prevents the IL12 from leaking into the blood.

Transmembrane and cytosolic domain
(SEQ ID NO: 12)
CTTGTGCTCTTTGGGGCAGGATTCGGCGCAGTAATAACAGTCGTCGTCA
TCGTTGTCATCATCAAATGCTTCTGTAAGCACAGAAGCTGTTTCAGAAG
AAATGAGGCAAGCAGAGAAACAAACAACAGCCTTACCTTCGGGCCTGAA
GAAGCATTAGCTGAACAGACCGTCTTCCTT
PD1-IL12-transmembrane domain construct
(SEQ ID NO: 13)
AGCGCCCAATACGCAAACCGCCTCTCCCCGCGCGTTGGCCGATTCATTA
ATGCAGCTGGCACGACAGGTTTCCCGACTGGAAAGCGGGCAGTGAGCGC
AACGCAATTAATGTGAGTTAGCTCACTCATTAGGCACCCCAGGCTTTAC
ACTTTATGCTTCCGGCTCGTATGTTGTGTGGAATTGTGAGCGGATAACA
ATTTCACACAGGAAACAGCTATGACCATGATTACGCCAAGCTCGAAATT
AACCCTCACTAAAGGGAACAAAAGCTGGAGCTCCACCGCGGTGGCGGCC
GCATAAACGCGTTTAAACAGTCCCCCGTACGCGGTACATCGTACGCACA
CTTCACTAACGATGTCGTACATCGATTACACAAAGAAGTAGAGTCATAC
GACGTACGTTTCCCTATAAAATCGGTAAACCTAGACGCGGTGTTTCTAT
CCATAAACGTAACACGTGTACGTCTACGTTGGAAGATACCCTTGACCGA
ACACAATCCTTATCAGACGGCCTACGGATGTTCTAACGACAGATTATAC
AGCTACAACGAGTACGCTTTTTCTCATTTAAAACAAGACCGTGTAAAGA
TCATAGAACTCCCATGTGACGACGATTACAGCGTCGTGTTAATCACACA
CGATAGCCGTTCGACTATTACACCGGATAAAGTGACCGGGTGGCTGCGC
ACGACCCGTCTACGTTACGTAAACGTATCCCTACCCAAGGGTTCCACGG
AAACGGGACACAACGTAACGTGTCTAACTCCCACACACGTCAATCTATG
TCATCGTTGTCGTATAACGATTACCAAAACGGGCGTGGACGCAACCGCG
TTCTCATGCGTCGACGGCGATACATGCACCGAACACGACACGACCGCGT
CAACGTGTACGATTATTATAAAAACGACGGGACTGGACTTTTTGTTTAT
GGGGAAACTCTAAAAAAAATTGTCAATTAAAGTAACTGCAGATCGATCG
CATATGAAAATTGAAATTTTATTTTTTTTTTTTGGAATATAAATAATGG
TGAGCAAGGGCGAGGAGGTCATCAAAGAGTTCATGCGCTTCAAGGTGCG
CATGGAGGGCTCCATGAACGGCCACGAGTTCGAGATCGAGGGCGAGGGC
GAGGGCCGCCCCTACGAGGGCACCCAGACCGCCAAGCTGAAGGTGACCA
AGGGCGGCCCCCTGCCCTTCGCCTGGGACATCCTGTCCCCCCAGTTCAT
GTACGGCTCCAAGGCGTACGTGAAGCACCCCGCCGACATCCCCGATTAC
AAGAAGCTGTCCTTCCCCGAGGGCTTCAAGTGGGAGCGCGTGATGAACT
TCGAGGACGGCGGTCTGGTGACCGTGACCCAGGACTCCTCCCTCCAAGA
CGGCACGCTGATCTACAAGGTGAAGATGCGCGGCACCAACTTCCCCCCC
GACGGCCCCGTAATGCAGAAGAAGACCATGGGCTGGGAGGCCTCCACCG
AGCGCCTGTACCCCCGCGACGGCGTGCTGAAGGGCGAGATCCACCAGGC
CCTGAAGCTGAAGGACGGCGGCCACTACCTGGTGGAGTTCAAGACCATC
TACATGGCCAAGAAGCCCGTGCAACTGCCCGGCTACTACTACGTGGACA
CCAAGCTGGACATCACCTCCCACAACGAGGACTACACCATCGTGGAACA
GTACGAGCGCTCCGAGGGCCGCCACCACCTGTTCCTGGGGCATGGCACC
GGCAGCACCGGCAGCGGCAGCTCCGGCACCGCCTCCTCCGAGGACAACA
ACATGGCCGTCATCAAAGAGTTCATGCGCTTCAAGGTGCGCATGGAGGG
CTCCATGAACGGCCACGAGTTCGAGATCGAGGGCGAGGGCGAGGGCCGC
CCCTACGAGGGCACCCAGACCGCCAAGCTGAAGGTGACCAAGGGCGGCC
CCCTGCCCTTCGCCTGGGACATCCTGTCCCCCCAGTTCATGTACGGCTC
CAAGGCGTACGTGAAGCACCCCGCCGACATCCCCGATTACAAGAAGCTG
TCCTTCCCCGAGGGCTTCAAGTGGGAGCGCGTGATGAACTTCGAGGACG
GCGGTCTGGTGACCGTGACCCAGGACTCCTCCCTCCAAGACGGCACGCT
GATCTACAAGGTGAAGATGCGCGGCACCAACTTCCCCCCCGACGGCCCC
GTAATGCAGAAGAAGACCATGGGCTGGGAGGCCTCCACCGAGCGCCTGT
ACCCCCGCGACGGCGTGCTGAAGGGCGAGATCCACCAGGCCCTGAAGCT
GAAGGACGGCGGCCACTACCTGGTGGAGTTCAAGACCATCTACATGGCC
AAGAAGCCCGTGCAACTGCCCGGCTACTACTACGTGGACACCAAGCTGG
ACATCACCTCCCACAACGAGGACTACACCATCGTGGAACAGTACGAGCG
CTCCGAGGGCCGCCACCACCTGTTCCTGTACGGCATGGACGAGCTGTAC
AAGTAACCCGGGAAAAATTGAAATTTTATTTTTTTTTTTTGGAATATAA
ATAACCATGTGTCCTCAGAAGCTAACCATCTCCTGGTTTGCCATCGTTT
TGCTGGTGTCTCCACTCATGGCCATGTGGGAGCTGGAGAAAGACGTTTA
TGTTGTAGAGGTGGACTGGACTCCCGATGCCCCTGGAGAAACAGTGAAC
CTCACCTGTGACACGCCTGAAGAAGATGACATCACCTGGACCTCAGACC
AGAGACATGGAGTCATAGGCTCTGGAAAGACCCTGACCATCACTGTCAA
AGAGTTTCTAGATGCTGGCCAGTACACCTGCCACAAAGGAGGCGAGACT
CTGAGCCACTCACATCTGCTGCTCCACAAGAAGGAAAATGGAATTTGGT
CCACTGAAATTTTAAAAAATTTCAAAAACAAGACTTTCCTGAAGTGTGA
AGCACCAAATTACTCCGGACGGTTCACGTGCTCATGGCTGGTGCAAAGA
AACATGGACTTGAAGTTCAACATCAAGAGCAGTAGCAGTTCCCCTGACT
CTCGGGCAGTGACATGTGGAATGGCGTCTCTGTCTGCAGAGAAGGTCAC
ACTGGACCAAAGGGACTATGAGAAGTATTCAGTGTCCTGCCAGGAGGAT
GTCACCTGCCCAACTGCCGAGGAGACCCTGCCCATTGAACTGGCGTTGG
AAGCACGGCAGCAGAATAAATATGAGAACTACAGCACCAGCTTCTTCAT
CAGGGACATCATCAAACCAGACCCGCCCAAGAACTTGCAGATGAAGCCT
TTGAAGAACTCACAGGTGGAGGTCAGCTGGGAGTACCCTGACTCCTGGA
GCACTCCCCATTCCTACTTCTCCCTCAAGTTCTTTGTTCGAATCCAGCG
CAAGAAAGAAAAGATGAAGGAGACAGAGGAGGGGTGTAACCAGAAAGGT
GCGTTCCTCGTAGAGAAGACATCTACCGAAGTCCAATGCAAAGGCGGGA
ATGTCTGCGTGCAAGCTCAGGATCGCTATTACAATTCCTCATGCAGCAA
GTGGGCATGTGTTCCCTGCAGGGTCCGATCCGGTGGCGGTGGCTCGGGC
GGTGGTGGGTCGGGTGGCGGCGGATCTAGGGTCATTCCAGTCTCTGGAC
CTGCCAGGTGTCTTAGCCAGTCCCGAAACCTGCTGAAGACCACAGATGA
CATGGTGAAGACGGCCAGAGAAAAACTGAAACATTATTCCTGCACTGCT
GAAGACATCGATCATGAAGACATCACACGGGACCAAACCAGCACATTGA
AGACCTGTTTACCACTGGAACTACACAAGAACGAGAGTTGCCTGGCTAC
TAGAGAGACTTCTTCCACAACAAGAGGGAGCTGCCTGCCCCCACAGAAG
ACGTCTTTGATGATGACCCTGTGCCTTGGTAGCATCTATGAGGACTTGA
AGATGTACCAGACAGAGTTCCAGGCCATCAACGCAGCACTTCAGAATCA
CAACCATCAGCAGATCATTCTAGACAAGGGCATGCTGGTGGCCATCGAT
GAGCTGATGCAGTCTCTGAATCATAATGGCGAGACTCTGCGCCAGAAAC
CTCCTGTGGGAGAAGCAGACCCTTACAGAGTGAAAATGAAGCTCTGCAT
CCTGCTTCACGCCTTCAGCACCCGCGTCGTGACCATCAACAGGGTGATG
GGCTATCTGAGCTCCGCCCTTGTGCTCTTTGGGGCAGGATTCGGCGCAG
TAATAACAGTCGTCGTCATCGTTGTCATCATCAAATGCTTCTGTAAGCA
CAGAAGCTGTTTCAGAAGAAATGAGGCAAGCAGAGAAACAAACAACAGC
CTTACCTTCGGGCCTGAAGAAGCATTAGCTGAACAGACCGTCTTCCTTT
GAGAATTCACGAATCGAATAAAAACCCGTGTACACACGGACGTTAATTT
TTTTTGTGGTTTAAAAAATGACCACATTTACGCTTTTTTTTAACGCGTT
ATATAAGGTATCTCGTTTGTCTATAACAAAGATCGTAACTGACCTTTTT
TATATCGAGAAAACATACGTTTAGTTCATCCTCAAACGTAACACCGTAA
CTGCCTCGGACATCCTCCTTGTTGTCGTACACAAACATACTAATCGGAT
GCGTGAAATGAGGATTCACTTTAATCGGATTGGTTTCTAGGTTAACACA
TGTTACACAAGATCCTAAGATGGTTATGGACACATCCTTGTTGTGATGT
AACGAGTCGGGAAGTTGATTGCCGTAGTTGCCCACGTCGCCCTCCGGTT
CCAGACACGTAATGGTTAGGTATATATCCGAATACTTCGTCAACGGATG
AGTCGTAAATAACATGATGGATAGCTTGTTCCCATCTCCTGCACCAGCA
CTGGCCGCCACAAATCGTTGTACCACGTTAGTAATCGTAATGTTTATCA
TAAGCCCGTACCCGGTTAATATGAGCGTGGACGTTTTATGATCGTATCG
TTCCTTCATGTGACATTCTCCCATAACCGTTTCGACGTACCGATTTAAC
CCGATGGTTAGCTCGGCGGCTAAGTGCCAGTGGATCCCCCAATTCGATA
TCAAGCTTATCGATACCGTCGACCTCGAGGGGGGGCCCGGTACCCAATT
CGCCCTATAGTGAGTCGTATTACAATTCACTGGCCGTCGTTTTACAACG
TCGTGACTGGGAAAACCCTGGCGTTACCCAACTTAATCGCCTTGCAGCA
CATCCCCCTTTCGCCAGCTGGCGTAATAGCGAAGAGGCCCGCACCGATC
GCCCTTCCCAACAGTTGCGCAGCCTGAATGGCGAATGGGACGCGCCCTG
TAGCGGCGCATTAAGCGCGGCGGGTGTGGTGGTTACGCGCAGCGTGACC
GCTACACTTGCCAGCGCCCTAGCGCCCGCTCCTTTCGCTTTCTTCCCTT
CCTTTCTCGCCACGTTCGCCGGCTTTCCCCGTCAAGCTCTAAATCGGGG
GCTCCCTTTAGGGTTCCGATTTAGTGCTTTACGGCACCTCGACCCCAAA
AAACTTGATTAGGGTGATGGTTCACGTAGTGGGCCATCGCCCTGATAGA
CGGTTTTTCGCCCTTTGACGTTGGAGTCCACGTTCTTTAATAGTGGACT
CTTGTTCCAAACTGGAACAACACTCAACCCTATCTCGGTCTATTCTTTT
GATTTATAAGGGATTTTGCCGATTTCGGCCTATTGGTTAAAAAATGAGC
TGATTTAACAAAAATTTAACGCGAATTTTAACAAAATATTAACGCTTAC
AATTTAGGTGGCACTTTTCGGGGAAATGTGCGCGGAACCCCTATTTGTT
TATTTTTCTAAATACATTCAAATATGTATCCGCTCATGAGACAATAACC
CTGATAAATGCTTCAATAATATTGAAAAAGGAAGAGTATGAGTATTCAA
CATTTCCGTGTCGCCCTTATTCCCTTTTTTGCGGCATTTTGCCTTCCTG
TTTTTGCTCACCCAGAAACGCTGGTGAAAGTAAAAGATGCTGAAGATCA
GTTGGGTGCACGAGTGGGTTACATCGAACTGGATCTCAACAGCGGTAAG
ATCCTTGAGAGTTTTCGCCCCGAAGAACGTTTTCCAATGATGAGCACTT
TTAAAGTTCTGCTATGTGGCGCGGTATTATCCCGTATTGACGCCGGGCA
AGAGCAACTCGGTCGCCGCATACACTATTCTCAGAATGACTTGGTTGAG
TACTCACCAGTCACAGAAAAGCATCTTACGGATGGCATGACAGTAAGAG
AATTATGCAGTGCTGCCATAACCATGAGTGATAACACTGCGGCCAACTT
ACTTCTGACAACGATCGGAGGACCGAAGGAGCTAACCGCTTTTTTGCAC
AACATGGGGGATCATGTAACTCGCCTTGATCGTTGGGAACCGGAGCTGA
ATGAAGCCATACCAAACGACGAGCGTGACACCACGATGCCTGTAGCAAT
GGCAACAACGTTGCGCAAACTATTAACTGGCGAACTACTTACTCTAGCT
TCCCGGCAACAATTAATAGACTGGATGGAGGCGGATAAAGTTGCAGGAC
CACTTCTGCGCTCGGCCCTTCCGGCTGGCTGGTTTATTGCTGATAAATC
TGGAGCCGGTGAGCGTGGGTCTCGCGGTATCATTGCAGCACTGGGGCCA
GATGGTAAGCCCTCCCGTATCGTAGTTATCTACACGACGGGGAGTCAGG
CAACTATGGATGAACGAAATAGACAGATCGCTGAGATAGGTGCCTCACT
GATTAAGCATTGGTAACTGTCAGACCAAGTTTACTCATATATACTTTAG
ATTGATTTAAAACTTCATTTTTAATTTAAAAGGATCTAGGTGAAGATCC
TTTTTGATAATCTCATGACCAAAATCCCTTAACGTGAGTTTTCGTTCCA
CTGAGCGTCAGACCCCGTAGAAAAGATCAAAGGATCTTCTTGAGATCCT
TTTTTTCTGCGCGTAATCTGCTGCTTGCAAACAAAAAAACCACCGCTAC
CAGCGGTGGTTTGTTTGCCGGATCAAGAGCTACCAACTCTTTTTCCGAA
GGTAACTGGCTTCAGCAGAGCGCAGATACCAAATACTGTTCTTCTAGTG
TAGCCGTAGTTAGGCCACCACTTCAAGAACTCTGTAGCACCGCCTACAT
ACCTCGCTCTGCTAATCCTGTTACCAGTGGCTGCTGCCAGTGGCGATAA
GTCGTGTCTTACCGGGTTGGACTCAAGACGATAGTTACCGGATAAGGCG
CAGCGGTCGGGCTGAACGGGGGGTTCGTGCACACAGCCCAGCTTGGAGC
GAACGACCTACACCGAACTGAGATACCTACAGCGTGAGCTATGAGAAAG
CGCCACGCTTCCCGAAGGGAGAAAGGCGGACAGGTATCCGGTAAGCGGC
AGGGTCGGAACAGGAGAGCGCACGAGGGAGCTTCCAGGGGGAAACGCCT
GGTATCTTTATAGTCCTGTCGGGTTTCGCCACCTCTGACTTGAGCGTCG
ATTTTTGTGATGCTCGTCAGGGGGGCGGAGCCTATGGAAAAACGCCAGC
AACGCGGCCTTTTTACGGTTCCTGGCCTTTTGCTGGCCTTTTGCTCACA
TGTTCTTTCCTGCGTTATCCCCTGATTCTGTGGATAACCGTATTACCGC
CTTTGAGTGAGCTGATACCGCTCGCCGCAGCCGAACGACCGAGCGCAGC
GAGTCAGTGAGCGAGGAAGCGGAAG

### Example 2—Generation of vMYX-TIM3 Constructs

vTIM3 was generated by homologously recombining unmodified myxoma virus (strain Lausanne) with pBS-M135/M136-sE/L GFP+TIM3, a plasmid which contains the following critical elements.
pBluescript plasmid backbone
Region of myxoma genome homologous to M134/M135
eGFP driven by the consensus poxviral synthetic early/late promoter
aa1-195 of murine TIM3 driven by a second consensus poxviral synthetic early/late promoter
Region of myxoma genome homologous to M136

TIM3-GFP Construct
(SEQ ID NO: 10)
NNNNNNNNNNCNCGNGGNGGCGGCCGCTCTAGTAGGATTACCTGGTCTA
TATAGATAACAAAACCTACGTACGTATAAACGAGACCGTTGTACCGGAG
AACGAGTATCTGGCAGCGAAGGCCCCGCGAGTGACCTGTTTCCACACGG
ACTTGATCCCCATTACGGACGAAGAGACACAACGACGTTTTGAGAAAAT
GATTGTACAGGCGGCGTTAGAGGACGCCCTAACGAGCATCTTTGAGGAG
CACGACAATAACGTAACCGATTACTTCGCGGAATACATGCGATCCCTCC
AAATGGCGAATAAAAGTCATACGAATAATATTATCGCGGTCGCTTTAGC
GGGGATAATCGTCATTGTAACGACCTACGTGTTTACTAGATTACGCACT
AAGCAAAAAAAAGGAAATTATAACGTACGTAATAAGATAGATAATTCCA
TACAGAAAGAGATTCAGTTGGACGGTGTATATACTACTGACAACGTTTT
TATATAAACATGGTGTTTATATTTATTATCACCTGTGTATGTTTGGTGA
CGAGATCCTGTGGGGGTGGGTTAGAAGACGATATAGATCGCATATTTCA
AAAACGATACAACGAACTGAGCCAGCCGATTAANNNCAATATGCGTACA
CTGTGCAAGTTTAGAGGAATTACCGCGACTATGTTTACGGAAGGAGAAT
CTTACCTTATTCAATGTCCCATAATTCACGATTACGTGCTACGGGCGCT
GTATGACTTAGTGGAAGGAAGTTACACGGTACGCTGGGAACGCGAAACG
GAAGACGATGTTGAGTCGGTAGATCCGAAGTTAGTCAAAGGGACGCTAT
TATACCTCCAACCTAACGCGTCCAGTATAGGAACGTATCTATGTACCTT
ACACGATAACCGAGGTATGTGTTATCAATCTGTCGCGCACGTCATCCGA
CGTCCGAAGATGCAATGCGTGAAACATGCACATACGACATCGGACAGCA
ACCTGTGGATATACCTCGCCATTTTAGCAGTTTTGATATCCTTAGGCGT
CCTGTAAAGGAAACGCGCCAGACTCCGGAACTATGAAGGATTTATCACT
GTATACAGACTCCGACGTACGAAGGATAATCACGACGTAACTCGAACTC
TGCAGGTCGACTCTAGAGGATCTACTAGTCATATGGATTTAAAAATAGC
GGAGCTTAAAAATTGAAATTTTATTTTTTTTTTTTGGAATATAAATAAG
CTCGAAGTCGACAGATCTAGGCCTGGTACCCGATCCACCGGTCGCCACC
ATGGTGAGCAAGGGCGAGGAGCTGTTCACCGGGGTGGTGCCCATCCTGG
TCGAGCTGGACGGCGACGTAAACGGCCACAAGTTCAGCGTGTCCGGCGA
GGGCGAGGGCGATGCCACNTACGGCAAGCTGACCCTGAAGTTCATCTGC
ACCACCGGCAAGCTGCCCGTGCCCTGGCCCACCCTCGTGACCACCCTGA
CCTACGGCGTGCAGTGCTTCAGCCGCTACCCCGACCACATGAAGCAGCA
CGACTTCTTCAAGTCCGCCATGCCCGAAGGCTACGTCCAGGAGCGCACC
ATCTTCTTCAAGGACGACGGCAACTACAAGACCCGCGCCGAGGTGAAGT
TCGAGGGCGACACCCTGGTGAACCGCATCGAGCTGAAGGGCATCGACTT
CAAGGAGGACGGCAACATCCTGGGGCACAAGCTGGAGTACAACTACAAC
AGCCACAACGTCTATATCATGGCCGACAAGCAGAAGAACGGCATCAAGG
TGAACTTCAAGATCCGCCACAACATCGAGGACGGCAGCGTGCAGCTCGC
CGACCACTACCAGCAGAACACCCCCATCGGCGACGGCCCCGTGCTGNTG
CCCGACAACCACTACCTGAGCACCCAGTCCGCCCTGAGCAAAGACCCCA
ACGAGAAGCGCGATCACATGGTCCTGCTGGAGTTCGTGACCGCCGCCGG
GATCACTCTCGGCATGGACGAGCTGTACAAGTAAAGCGGCCGGGTAATT
ACCCGGGATGTTTTCAGGTCTTACCCTCAACTGTGTCCTGCTGCTGCTG
CAACTACTACTTGCAAGGTCATTGGAAAATGCTTATGTGTTTGAGGTTG
GTAAGAATGCCTATCTGCCCTGCAGTTACACTCTATCTACACCTGGGGC
ACTTGTGCCTATGTGCTGGGGCAAGGGATTCTGTCCTTGGTCACAGTGT
ACCAACGAGTTGCTCAGAACTGATGAAAGAAATGTGACATATCAGAAAT
CCAGCAGATACCAGCTAAAGGGCGATCTCAACAAAGGAGACGTGTCTCT
GATCATAAAGAATGTGACTCTGGATGACCATGGGACCTACTGCTGCAGG
ATACAGTTCCCTGGTCTTATGAATGATAAAAAATTAGAACTGAAATTAG
ACATCAAAGCAGCCAAGGTCACTCCAGCTCAGACTGCCCATGGGGACTC
TACTACAGCTTCTCCAAGAACCCTAACCACGGAGAGAAATGGTTCAGAG
ACACAGACACTGGTGACCCTCCATAATAACAATGGAACAAAAATTTCCA
CATGGGCTGATGAAATTAAGGACTCTGGAGAAACGATCAGAACTGCTAT
CCACTAGGAATTCTAACATTTTTTAAAACAATTTCGTTATGTTAAATTA
TGGAACGGTCGCCCACTTACACGGTACACGATAAACGCTTTTCTATCGT
CGCACTAAACGGACAATACGACATGGTGGACGATTTTGGTCTTAGTTTT
TCTTACACAGCGATCGACGATATTTCTAAAAATCATTCCATCAAACACG
TTTTAGAAGAATACTTTTCATGGCGCGCGTATATAGGCCGGGTATGTAT
CATACCGAATCACGTGGGAAAGCTCTACATCAAACTTACAAAGTTGGAC
ACCACGGCGAAGAACAAACTAGGCAATCTAGATATATTGTTATGCGACG
TGTTAAAAATAGACGAGGACGGAGGCAACGAGAAACTGTTTCAATTCAT
ACGGTCGCGGATCCCCCAATTCGATATCAAGCTTATCGATACCGTCGAC
CTCG
Soluble TIM3
(SEQ ID NO: 11)
ATGTTTTCAGGTCTTACCCTCAACTGTGTCCTGCTGCTGCTGCAACTAC
TACTTGCAAGGTCATTGGAAAATGCTTATGTGTTTGAGGTTGGTAAGAA
TGCCTATCTGCCCTGCAGTTACACTCTATCTACACCTGGGGCACTTGTG
CCTATGTGCTGGGGCAAGGGATTCTGTCCTTGGTCACAGTGTACCAACG
AGTTGCTCAGAACTGATGAAAGAAATGTGACATATCAGAAATCCAGCAG
ATACCAGCTAAAGGGCGATCTCAACAAAGGAGACGTGTCTCTGATCATA
AAGAATGTGACTCTGGATGACCATGGGACCTACTGCTGCAGGATACAGT
TCCCTGGTCTTATGAATGATAAAAAATTAGAACTGAAATTAGACATCAA
AGCAGCCAAGGTCACTCCAGCTCAGACTGCCCATGGGGACTCTACTACA
GCTTCTCCAAGAACCCTAACCACGGAGAGAAATGGTTCAGAGACACAGA
CACTGGTGACCCTCCATAATAACAATGGAACAAAAATTTCCACATGGGC
TGATGAAATTAAGGACTCTGGAGAAACGATCAGAACTGCTATCCAC

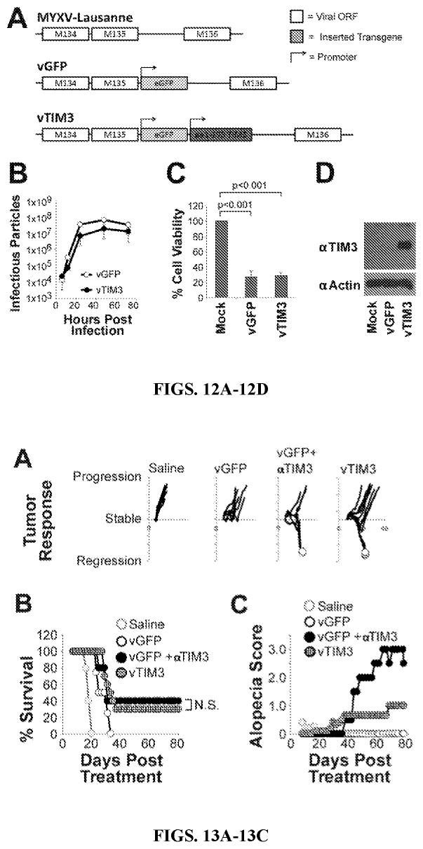

pBS-M135/M136-sE/L GFP+TIM3 was transfected into BSC40 cells which were then infected with unmodified myxoma virus (strain Lausanne). Cells were cultured for 72 hours which produces recombinant viruses in which the untranslated region of the viral genome between M135 and M136 is replaced by a cassette expressing both eGFP and soluble TIM3 (FIG. 12A). Recombinant virus was then quadruple plaque purified on BSC40 cells by selecting GFP⁺clones. Clonality of the final virus (vTIM3) was then confirmed using PCR.

In Vitro Characterization of vTIM3: vTIM3 secretes soluble PD1 from infected cells: Secretion of soluble PD1 from virally infected cells was confirmed by western blotting supernatants from B16/F10 melanoma cells infected with either saline (mock), vGFP (control virus), or vTIM3 after 24 hours of infection. A strong band consistent with the soluble portion of TIM3 was observed specifically in the supernatant of cell infected with vTIM3 (FIG. 12D).

vTIM3 displays normal replication and oncolytic capacity in vitro: To determine whether insertion of the TIM3 transgene would alter MYXV replication, single step growth curves were performed on both vGFP and vTIM3 in a variety of cells. It was observed that both viruses displayed identical replication in all tested cell types (FIGS. 12B and 12C). To further test whether secretion of the TIM3 transgene would alter MYXV's ability to kill directly infected cells, it was next asked how effective both vGFP and vTIM3 were at killing B16/F10 melanoma cells. B16/F10 cells were infected with either vGFP or vTIM3 at the indicated multiplicities of infection. After 24 hours, cellular viability was analyzed using MTT assay. It was observed that both vGFP and vTIM3 displayed an identical capacity to kill infected melanoma cell in vitro (FIG. 13).

Oncolytic potential against melanoma in vivo: To test whether vTIM3 displayed increased oncolytic capacity in vivo, its ability to regress established melanoma tumors was tested in mice. C57/B6 mice were implanted subcutaneously with 5×10⁵ B16/F10 melanoma cells. Treatment was initiated seven days after injection of tumor cells (when tumors are approximately 15-20 mm²). Treatment consisted of two intratumoral injections of either saline, 1×10⁷ FFU of vGFP, or 1×10⁷ FFU of vTIM3 given on days 7 and 12. Animals were then monitored daily for tumor size and euthanized when tumors reached 150mm in any direction. Animals treated with vGFP displayed reduced tumor growth, however, the majority of tumors in these animals still progressed eventually requiring euthanasia (FIG. 13). In contrast, many mice treated with vTIM3 displayed rapid regression of established tumors resulting in complete durable remissions in 7/12 animals.

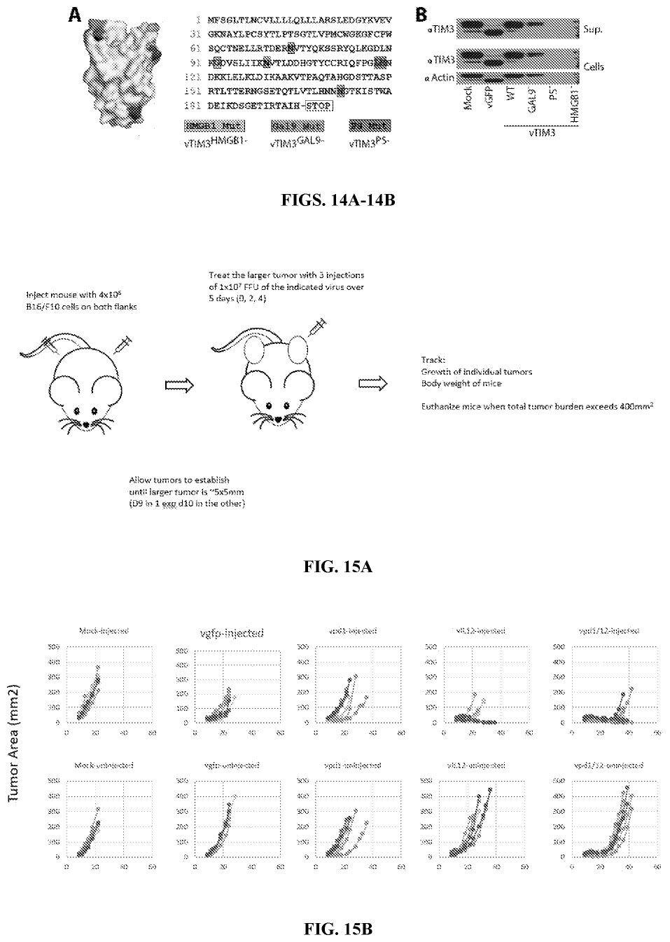

A series of viruses were constructed expressing variants of the soluble TIM3 protein in which previously validated binding sites for each TIM3 ligand have been removed through mutagenesis (FIG. 14). Each of these viruses are tested for their ability to induce atumor immunity and eradicate established tumors in vivo. This identifies the mechanisms involved in vTIM3-based checkpoint blockade as well as by allowing for the construction of a next generation vTIM3 construct with improved therapeutic efficacy.

### Example 3—vMYX-Therapy in Metastatic Disease

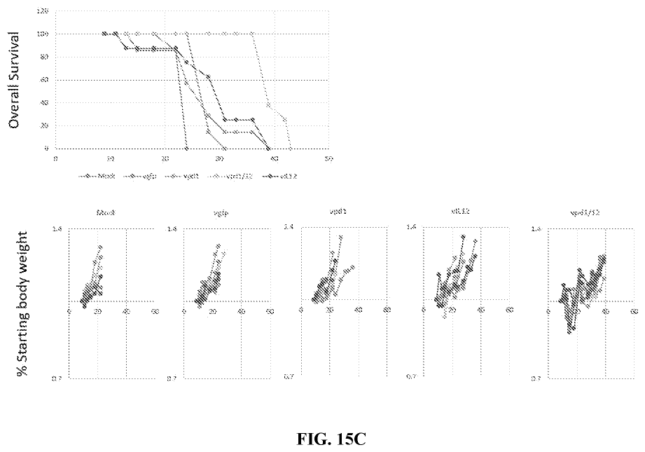

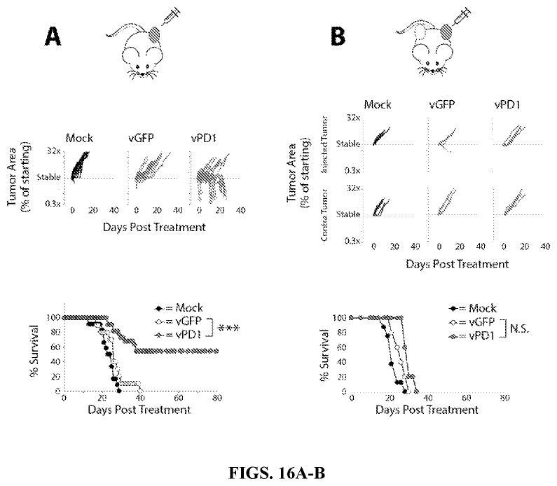

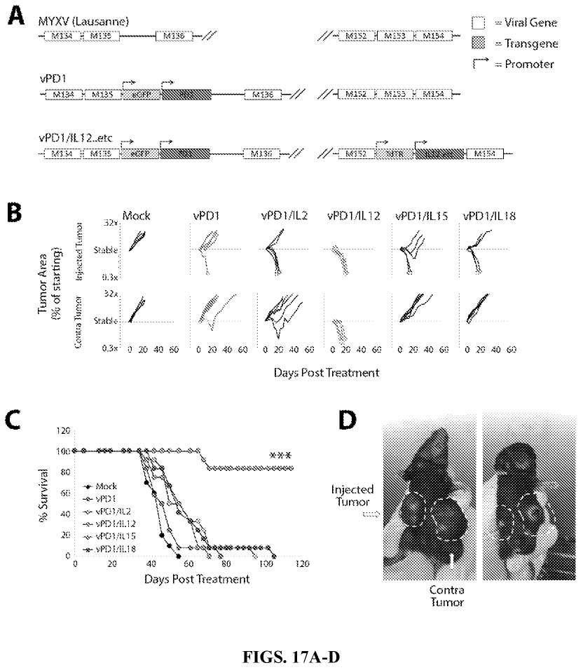

A recombinant MYXV which expresses a secreted form of soluble PD1 (vPD1) was also studied to determine its activity relative to metastatic cancers. Unfortunately, while vPD1 is extremely effective at eradicating localized disease (e.g., through the maintenance of anti-tumor immunity) see, e.g., FIG. 16, additional experiments have indicated that it has reduced effectiveness relative to non-injected, metastatic tumors (see FIG. 16B). Due to the inability of vPD1 to effectively regress non-treated lesions, to advance the clinical potential of this virus additional modifications were studied that could be added to the vPD1 backbone which would result in improved systemic efficacy. Additional recombinant viruses which expressed both soluble PD1 and either additional soluble T cell checkpoint proteins or a series of proinflammatory cytokines (see, FIG. 17A). Each of these viruses was then purified to clonality and tested for its ability to regress both injected and non-injected Lewis Lung Carcinomas (LLC) in a standard contralateral tumor model. The results indicated that, of the molecules tested, only inclusion of IL-12 (encoded as a p40/p35 fusion protein) significantly improved efficacy of the vPD1 backbone against non-injected lesions (FIG. 17B-C). Impressively, however, vPD1/IL12 was able to fully regress both injected and non-injected lesions in virtually all treated mice (durable complete response lasting >120 days in 10/12 animals) resulting in an effective ‘cure rate’ of almost 90%. Remarkably, even in animals with bulky, well-established disease (animals from the initial ‘mock’ cohort displaying a total tumor burden between 350-400mm²), treatment of a single tumor with vPD1/IL12 could cause both complete elimination of the treated tumors as well as significant regression in the non-treated tumors (FIG. 17D). These studies demonstrate that vPD1/IL12 virus represented a novel therapeutic agent with strong clinical potential against even late stage, metastatic disease.

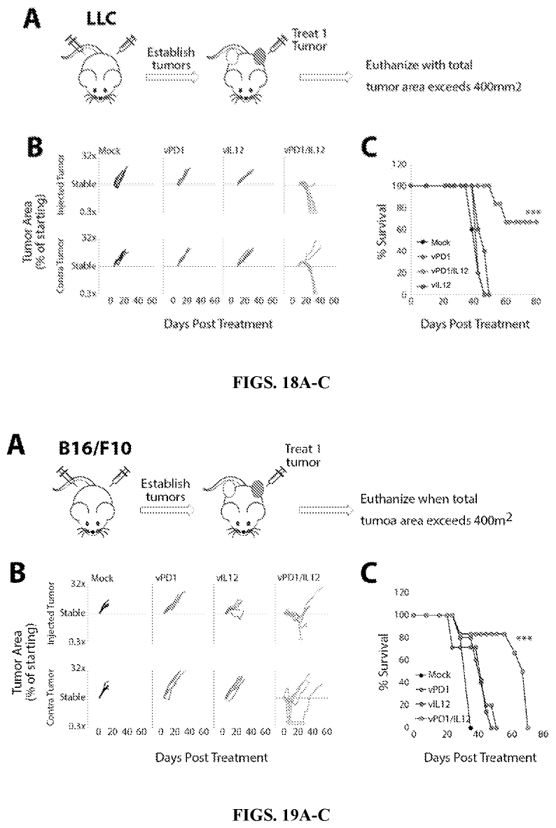

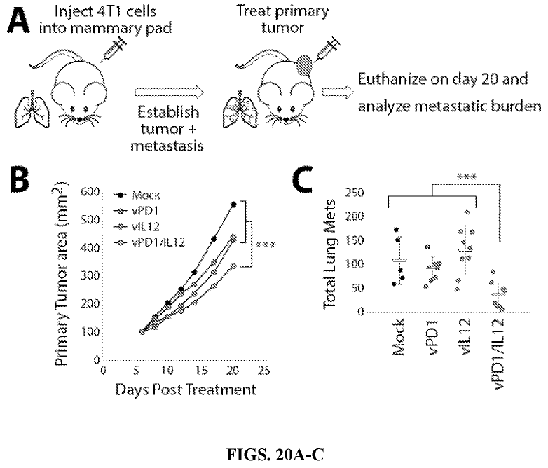

To advance on this exciting initial finding, the therapeutic breadth of the vPD1/IL12 virus was further studied. To accomplish this, a single recombinant MYXV expressing an IL12 fusion protein (vIL12) was compared the efficacy of this virus to that of both vPD1 and vPD1/IL12 in preclinical models of metastatic disease: LLC lung cancer (SQ contralateral tumor model), B16/F10 melanoma (SQ contralateral tumor model), 4T1 and triple negative breast cancer (single SQ tumor spontaneously metastatic to the lung). The results from all models clearly demonstrated that: 1) the vPD1/IL12 virus was capable of regressing both treated and non-treated lesions from a wide range of different malignancies including tumors representing both immunologically ‘hot’ (LLC and B16/F10) and immunologically ‘cold’ (4T1 and ID8) forms of disease. 2) This clinical efficacy was not observed in any model following treatment with either singly recombinant virus (vPD1 or vIL12) indicating that that vPD1/IL12′s therapeutic effect is due to a unique form of combinatorial synergy (FIGS. 18-20).

All of the methods disclosed and claimed herein can be made and executed without undue experimentation in light of the present disclosure. While the compositions and methods of this disclosure have been described in terms of preferred embodiments, it will be apparent to those of skill in the art that variations may be applied to the methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### REFERENCES

The following references, to the extent that they provide exemplary procedural or other details supplementary to those set forth herein, are specifically incorporated herein by reference.
Ausubel et al., Current Protocols in Molecular Biology, Greene Publ. Assoc. Inc. & John Wiley & Sons, Inc., MA, 1996.
Bartee et al., 2017. Cancer Research 77, 2952-2963.
Cameron et al., Virology, 264:298-318, 1999.
Chen and Okayama, Mol. Cell Biol., 7(8):2745-2752, 1987.
Cheng et al. “Membrane-Tethered Proteins for Basic Research, Imaging, and Therapy” Medicinal Research Reviews, Vol. 28, No. 6, 885-928, 2008.
Fechheimer et al., Proc Natl. Acad. Sci. USA, 84:8463-8467, 1987.
Fraley et al., Proc. Natl. Acad. Sci. USA, 76:3348-3352, 1979.
Frankel and Pabo, Cell, 55(6):1189-1193, 1988.
Gopal, Mol. Cell Biol., 5:1188-1190, 1985.
Graham and Van Der Eb, Virology, 52:456-467, 1973.
Harland and Weintraub, J. Cell Biol., 101(3):1094-1099, 1985.
Johnson and Win, 2017. Oncoimmunology, Combination therapy with PD-1/PD-L1 blockade: An overview of ongoing clinical trials. DOI:10.1080/2162402X.2017.1408744
Johnston and McFadden, J. Virology, 77:6093-6100, 2003.
Kaeppler et al., Plant Cell Rep., 8:415-418, 1990.
Kaneda et al., Science, 243:375-378, 1989.
Kato et al, J. Biol. Chem., 266:3361-3364, 1991.
Kaufman, et al., 2015. Nature Rev. Drug Discov 14, 642-662.
Kerr and McFadden, Viral Immunology, 15:229-246, 2002.
Levin et al. (2012) Nature 484:529-533
Liu et al., J. Clin. Investig., 105:1613-1621, 2000.
Lucas and McFadden, J. Immunol., 173:4765-47742004.
Na et al., 2016. Cell Res 27:147-150.
Nabel et al., Science, 244(4910):1342-1344, 1989.
Nicolau and Sene, Biochim. Biophys. Acta, 721:185-190, 1982.
Nicolau et al., Methods Enzymol., 149:157-176, 1987.
Pardoll, 2012. Nature Reviews: Cancer 12:252-264.
PCT Application No. WO 04/078206.
PCT Application No. WO 94/09699.
PCT Application No. WO 95/06128.
Potrykus et al., Mol. Gen. Genet., 199(2):169-177, 1985.
Potter et al., Proc. Natl. Acad. Sci. USA, 81:7161-7165, 1984.
Rippe, et al., Mol. Cell Biol., 10:689-695, 1990.
Sambrook and Russell, Molecular Cloning: A Laboratory Manual, 3^{rd} Ed. Cold Spring Harbor Lab. Press, 2001.
Sambrook et al., In: Molecular Cloning: A Laboratory Manual, Vol. 1, Cold Spring Harbor Laboratory Press, Cold Spring Harbor, N.Y., (7)7:19-17.29, 1989.
Smallwood, S. E. et al., Curr Protoc Microbiol., Unit-14A.1, 2010.
Sypula et al., Gene Ther Mol Biol., 8:103-1142004.
Tan, et al., 2017. Nature Communications 8:14369-10.
Topalian, S. L., et al., 2015. Cancer Cell 27:450-461.
Tosic et al., PLoS One, e109801, 2014.
Tur-Kaspa et al., Mol. Cell Biol., 6:716-718, 1986.
U.S. Patent Application No. 20090035276.
U.S. Pat. No. 5,302,523
U.S. Pat. No. 5,322,783
U.S. Pat. No. 5,384,253
U.S. Pat. No. 5,464,765
U.S. Pat. No. 5,538,877
U.S. Pat. No. 5,538,880
U.S. Pat. No. 5,550,318
U.S. Pat. No. 5,563,055
U.S. Pat. No. 5,563,055
U.S. Pat. No. 5,580,859
U.S. Pat. No. 5,589,466
U.S. Pat. No. 5,591,616
U.S. Pat. No. 5,610,042
U.S. Pat. No. 5,656,610
U.S. Pat. No. 5,702,932
U.S. Pat. No. 5,736,524
U.S. Pat. No. 5,780,448
U.S. Pat. No. 5,789,215
U.S. Pat. No. 5,945,100
U.S. Pat. No. 5,981,274
U.S. Pat. No. 5,994,624
U.S. Pat. No. 8,613,915
Wilson et al., Science, 244:1344-1346, 1989.
Wong et al., Gene, 10:87-94, 1980.
Wu and Wu, Biochemistry, 27: 887-892, 1988.
Wu and Wu, J. Biol. Chem., 262:4429-4432, 1987.

What is claimed is:

1. A recombinant oncolytic virus comprising one or more expression cassettes encoding (a) a soluble form of programmed cell death protein 1 (PD1), and (b) interleukin 12 (IL-12) or interleukin 2 (IL-2), wherein the virus is replication competent.

2. The oncolytic virus of claim 1, wherein the virus comprises one or more expression cassettes encoding (a) a soluble form of programmed cell death protein 1 (PD1), and (b) interleukin 12 (IL-12).

3. A recombinant oncolytic virus comprising one or more expression cassettes encoding a mutant soluble form of PD1 (mutPD1) and optionally interleukin 12 (IL-12) or interleukin 2 (IL-2), wherein the virus is replication competent and wherein the mutPD1 prevents recognition of mutPD1 by an anti-PD1 antibody.

4. The oncolytic virus of claim 3, wherein the virus comprises one or more expression cassettes encoding a mutant soluble form of PD1 (mutPD1) and interleukin 12 (IL-12).

5. The oncolytic virus of claim 3, wherein the mutPD1 contains a mutation in the CD loop that prevents antibody recognition by anti-PD1 antibodies.

6. The oncolytic virus of claim 5, wherein the mutPD1 contains a point mutation in the CD loop comprising D85G.

7. The oncolytic virus of claim 6, wherein the mutPD1 is not recognized by pembrolizumab.

8. The oncolytic virus of claim 1 or 3, wherein the soluble PD1 comprises an extracellular region of human PD1.

9. The oncolytic virus of claim 8, wherein the extracellular region of human PD1 and IL-2 or IL-12 are encoded in a single expression cassette.

10. The oncolytic virus of claim 1 or 3, wherein the expression cassette(s) is/are under the control of one or more viral promoters.

11. The oncolytic virus of claim 10, wherein the one or more viral promoters is/are synthetic early/late poxvirus promoter.

12. The oncolytic virus of claim 11, wherein the synthetic early/late poxvirus promoter is at least 90% identical to SEQ ID NO: 20.

13. The oncolytic virus of claim 1 or 3, wherein the virus is selected from the group consisting of myxoma virus, reovirus, herpes simplex virus, Newcastle Disease virus, measles virus, retrovirus, poxvirus, rhabdovirus, picornavirus, coxsackievirus and parvovirus.

14. The oncolytic virus of claim 13, wherein the oncolytic virus is myxoma virus.

15. The oncolytic virus of claim 14, wherein the extracellular region of PD1, IL-2 and/or IL-12 expression cassette(s) is/are incorporated: (i) into the myxoma genome at the viral M153R open reading frame; or between the M135 and M136 genes.

16. The oncolytic virus of claim 14, wherein the extracellular region of PD1 is incorporated between the M135 and M136 genes.

17. The oncolytic virus of claim 14, wherein the extracellular region of IL-12 is incorporated into the myxoma genome at the viral M153R open reading frame.

18. The oncolytic virus of claim 15, further comprising a marker gene.

19. The oncolytic virus of claim 14, wherein IL-12 is fused to a transmembrane domain.

20. The oncolytic virus of claim 19, wherein the transmembrane domain is encoded by SEQ ID NO:12.

21. The oncolytic virus of claim 20, wherein the oncolytic virus is encoded by SEQ ID NO:13.

22. A pharmaceutical composition of the oncolytic virus of claim 1 or 3.

23. A method of treating a disease in a subject in need thereof comprising administering an effective amount of the oncolytic virus of claim 1 or 3, or the pharmaceutical composition of claim 22.

24. The method of claim 23, wherein the disease is cancer.

25. The method of claim 24, wherein the cancer has increased expression of programmed death-ligand 1 (PDL1).

26. The method of claim 24, wherein the subject has been determined to have a cancer that expresses increased PDL1.

27. The method of claim 24, wherein the cancer does not have increased expression of PDL 1.

28. The method of claim 24, wherein the cancer is melanoma, kidney cancer, colorectal cancer, breast cancer, lung cancer, head and neck cancer, brain cancer, leukemia, prostate cancer, bladder cancer, and ovarian cancer.

29. The method of claim 24, wherein the cancer is melanoma.

30. The method of claim 29, wherein the melanoma is metastatic melanoma.

31. The method of claim 23, wherein the oncolytic virus is administered intra-arterially, intravenously, intraperitoneally, or intratumorally.

32. The method of claim 23, wherein the oncolytic virus is administered two or more times.

33. The method of claim 23, further comprising administering at least a second anti-cancer therapy to the subject.

34. The method of claim 33, wherein the second anti-cancer therapy is administered concurrently or sequentially with the recombinant virus.

35. The method of claim 33, wherein the second anti-cancer therapy is an immunomodulator.

36. The method of claim 33, wherein the second anti-cancer therapy is immunotherapy, chemotherapy, radiotherapy, gene therapy, surgery, hormonal therapy, anti-angiogenic therapy or cytokine therapy.

37. The method of claim 36, wherein the immunotherapy is immune checkpoint inhibitor therapy.

38. The method of claim 37, wherein the immune checkpoint inhibitor therapy comprises treatment with an antibody directed to PD1, PDL1, or CTLA4.

39. The method of claim 38, wherein the antibody is Pembrolizumab, Nivolumab, Atezolizumab, Avelumab, Durvalumab, or Ipilimumab.

40. The method of claim 55, wherein the antibody is Pembrolizumab.

41. A method of treating a disease in a subject in need thereof comprising:
(a) testing the subject for overexpression of PDL1; and
(b) administering to a subject with increased expression of PDL1 a therapeutically effective amount of the oncolytic virus of claim 1 or 3.

42. The method of claim 41, wherein the disease is cancer.

43. The method of claim 42, wherein the cancer has increased expression of programmed death-ligand 1 (PDL1).

44. The method of claim 42, wherein the cancer does not have increased expression of PDL 1.

45. The method of claim 42, wherein the cancer is melanoma, kidney cancer, colorectal cancer, breast cancer, lung cancer, head and neck cancer, brain cancer, leukemia, prostate cancer, bladder cancer, and ovarian cancer.

46. The method of claim 42, wherein the cancer is melanoma.

47. The method of claim 46, wherein the melanoma is metastatic melanoma.

48. The method of claim 41, wherein the oncolytic virus is administered intra-arterially, intravenously, intraperitoneally, or intratumorally.

49. The method of claim 41, wherein the oncolytic virus is administered two or more times.

50. The method of claim 41, further comprising administering at least a second anti-cancer therapy to the subject.

51. The method of claim 50, wherein the second anti-cancer therapy is administered concurrently or sequentially with the recombinant virus.

52. The method of claim 50, wherein the second anti-cancer therapy is an immunomodulator.

53. The method of claim 50, wherein the second anti-cancer therapy is chemotherapy, immunotherapy, radiotherapy, gene therapy, surgery, hormonal therapy, anti-angiogenic therapy or cytokine therapy.

54. The method of claim 53, wherein the immunotherapy is immune checkpoint inhibitor therapy.

55. The method of claim 54, wherein the immune checkpoint inhibitor therapy comprises treatment with an antibody directed to PD1, PDL1, or CTLA4.

56. The method of claim 55, wherein the antibody is Pembrolizumab, Nivolumab, Atezolizumab, Avelumab, Durvalumab, or Ipilimumab.

57. A recombinant oncolytic virus comprising one or more expression cassettes encoding a soluble form of T-cell immunoglobulin and mucin-domain containing-3 (TIM3).

58. The oncolytic virus of claim 57, wherein the soluble TIM3 comprises an extracellular region of murine TIM3.

59. The oncolytic virus of claim 57, wherein the expression cassette(s) is/are under the control of one or more viral promoters.

60. The oncolytic virus of claim 60, wherein the one or more viral promoters is/are synthetic early/late poxvirus promoter.

61. The oncolytic virus of claim 57, wherein the virus is selected from the group consisting of myxoma virus, reovirus, herpes simplex virus, Newcastle Disease virus, measles virus, retrovirus, poxvirus, rhabdovirus, picornavirus, coxsackievirus and parvovirus.

62. The oncolytic virus of claim 61, wherein the oncolytic virus is myxoma virus.

63. The oncolytic virus of claim 62, wherein the is/are incorporated into the myxoma genome at the viral M153R open reading frame.

64. The oncolytic virus of claim 63, further comprising a marker gene.

65. The oncolytic virus of claim 64, wherein the marker gene is enhanced green fluorescent protein (eGFP).

66. A pharmaceutical composition of the oncolytic virus of claim 57.

67. A method of treating a disease in a subject in need thereof comprising administering an effective amount of the oncolytic virus of claim 57, or the pharmaceutical composition of claim 66.

68. The method of claim 67, wherein the disease is cancer.

69. The method of claim 74, wherein the cancer is melanoma, kidney cancer, colorectal cancer, breast cancer, lung cancer, head and neck cancer, brain cancer, leukemia, prostate cancer, bladder cancer, and ovarian cancer.

70. The method of claim 69, wherein the cancer is melanoma.

71. The method of claim 70, wherein the melanoma is metastatic melanoma.

72. The method of claim 71, wherein the oncolytic virus is administered intra-arterially, intravenously, intraperitoneally, or intratumorally.

73. The method of claim 72, wherein the oncolytic virus is administered two or more times.

74. The method of claim 73, further comprising administering at least a second anti-cancer therapy to the subject.

75. The method of claim 74, wherein the second anti-cancer therapy is administered concurrently or sequentially with the recombinant virus.

76. The method of claim 75, wherein the second anti-cancer therapy is an immunomodulator.

77. The method of claim 76, wherein the second anti-cancer therapy is immunotherapy, chemotherapy, radiotherapy, gene therapy, surgery, hormonal therapy, anti-angiogenic therapy or cytokine therapy.

78. The method of claim 77, wherein the immunotherapy is immune checkpoint inhibitor therapy.

79. The method of claim 78, wherein the immune checkpoint inhibitor therapy comprises treatment with an antibody directed to PD1, PDL1, or CTLA4.

80. The method of claim 79, wherein the antibody is Pembrolizumab, Nivolumab, Atezolizumab, Avelumab, Durvalumab, or Ipilimumab.

81. A method of treating a disease in a subject in need thereof comprising:
(a) testing the subject for overexpression of GAL9; and
(b) administering to a subject with increased expression of GAL9 a therapeutically effective amount of the oncolytic virus of claim 57.

82. The method of claim 81, wherein the disease is cancer.

83. The method of claim 90, wherein the cancer is melanoma, kidney cancer, colorectal cancer, breast cancer, lung cancer, head and neck cancer, brain cancer, leukemia, prostate cancer, bladder cancer, and ovarian cancer.

84. The method of claim 83, wherein the cancer is melanoma.

85. The method of claim 84, wherein the melanoma is metastatic melanoma.

86. The method of claim 81, wherein the oncolytic virus is administered intra-arterially, intravenously, intraperitoneally, or intratumorally.

87. The method of claim 81, wherein the oncolytic virus is administered two or more times.

88. The method of claim 81, further comprising administering at least a second anti-cancer therapy to the subject.

89. The method of claim 88, wherein the second anti-cancer therapy is administered concurrently or sequentially with the recombinant virus.

90. The method of claim 88, wherein the second anti-cancer therapy is an immunomodulator.

91. The method of claim 88, wherein the second anti-cancer therapy is chemotherapy, immunotherapy, radiotherapy, gene therapy, surgery, hormonal therapy, anti-angiogenic therapy or cytokine therapy.

92. The method of claim 91, wherein the immunotherapy is immune checkpoint inhibitor therapy.

93. The method of claim 92, wherein the immune checkpoint inhibitor therapy comprises treatment with an antibody directed to PD1, PDL1, or CTLA4.

94. The method of claim 93, wherein the antibody is Pembrolizumab, Nivolumab, Atezolizumab, Avelumab, Durvalumab, or Ipilimumab.

95. The method of claim 93, wherein the antibody is Pembrolizumab.
